(19) United States
(12) Patent Application Publication
(10) Pub. No.: US 2024/0338818 A1
(43) Pub. Date: Oct. 10, 2024

(54) DEVICES, SYSTEMS, AND METHODS FOR BONE BALANCE ADJUSTMENT BASED ON OSTEOPHYTE DETECTION

(71) Applicant: MAKO Surgical Corporation, Weston, FL (US)

(72) Inventor: James BONO, Wesston, FL (US)

(73) Assignee: MAKO Surgical Corporation, Weston, FL (US)

(21) Appl. No.: 18/622,218

(22) Filed: Mar. 29, 2024

Related U.S. Application Data
(60) Provisional application No. 63/597,845, filed on Nov. 10, 2023.
(60) Provisional application No. 63/494,586, filed on Apr. 6, 2023.

(51) Int. Cl. G06T 7/00

(52) U.S. Cl. CPC G06T 7/0012; G06T 2207/10081; G06T 2207/30008

(57) ABSTRACT

A method of assessing a joint may include identifying a first osteophyte in an image of the joint, identifying a cross-sectional area of the first osteophyte, executing an algorithm to determine one or more adjustment parameters based on the identified cross-sectional area of the first osteophyte, and outputting the one or more determined adjustment parameters to a display. The first osteophyte may be positioned under a soft tissue. The algorithm may apply an equation that receives, the identified cross-sectional area, and outputs the one or more adjustment parameters. The one or more adjustment parameters may include a predicted change in soft tissue laxity after the identified first osteophyte is removed, an adjustment to a planned bone resection depth of the one or more bone cuts, an adjustment to a planned bone resection angle of the one or more bone cuts, and/or an adjustment to a planned thickness of the implant.

### CROSS REFERENCE TO RELATED APPLICATION

This application claims priority to U.S. Provisional Application No. 63/597,845, filed on Nov. 10, 2023, and U.S. Provisional Application No. 63/494,586, filed on Apr. 6, 2023. The disclosures of the aforementioned priority applications are incorporated herein by reference in their entireties.

### FIELD OF THE DISCLOSURE

The present disclosure relates to systems, devices, and methods for optimizing medical procedures, and in particular to systems, devices, and methods for detecting osteophytes to optimize outcomes after joint replacement procedures, among other aspects.

### BACKGROUND OF THE DISCLOSURE

Surgeries incorporating prosthetics and/or implants, such as joint replacement procedures, often require careful consideration of various factors. For example, when cutting or resecting a bone for placement of a prosthesis, osteophytes may need to be removed, which may change a predicted result of the initially planned bone cut. Improved systems and methods for performing, collecting, and analyzing image data and/or osteophyte data are desired to help in planning surgeries and improve patient outcomes.

### BRIEF SUMMARY OF THE DISCLOSURE

In an aspect of the present disclosure, a method of assessing a joint may include identifying a first osteophyte in an image of the joint, identifying a cross-sectional area of the first osteophyte, executing an algorithm to determine one or more adjustment parameters based on the identified cross-sectional area of the first osteophyte, and outputting the one or more determined adjustment parameters to a display. The first osteophyte may be positioned under a soft tissue. The algorithm may apply a linear equation that receives, as input, the identified cross-sectional area, and output the one or more adjustment parameters. The one or more adjustment parameters include a predicted change in soft tissue laxity after the identified first osteophyte is removed, an adjustment to a planned bone resection depth of the one or more bone cuts, an adjustment to a planned bone resection angle of the one or more bone cuts, and/or an adjustment to a planned thickness of the implant.

Identifying the first osteophyte and/or identifying the cross-sectional area of the first osteophyte may include analyzing the image using one or more image processing techniques.

Identifying the cross-sectional area of the first osteophyte may include analyzing a first dimension of the first osteophyte and a second dimension of the first osteophyte. Identifying the cross-sectional area of the first osteophyte may include disregarding a third dimension of the first osteophyte.

The one or more adjustment parameters may include an adjustment to a planned bone resection depth of the one or more bone cuts and an adjustment to a planned bone resection angle of the one or more bone cuts.

Identifying the cross-sectional area of the first osteophyte may include determining that the image of the joint is an image of a set of images showing a greatest extent of the first osteophyte in a first dimension.

The linear relationship may include a linear relationship between the identified cross-sectional area and the adjustment to the planned bone resection depth such that, the greater the identified cross-sectional area, the greater the decrease in planned bone resection depth; and/or a linear relationship between the identified cross-sectional area and the planned thickness of the implant such that, the greater the identified cross-sectional area, the greater the increase to the planned thickness of the implant.

The method may further include identifying a second osteophyte in the image of the joint that is positioned under the soft tissue, identifying a cross-sectional area of the second osteophyte, identifying a position of the first osteophyte, and identifying a position of the second osteophyte. Executing the algorithm to determine the one or more adjustment parameters may be further based on the identified cross-sectional area of the second osteophyte, the identified position of the first osteophyte, and the identified position of the second osteophyte.

The method may further include determining, based on the identified position of the first osteophyte and the identified position of the second osteophyte, the first osteophyte is provided at a first side of the joint and the second osteophyte is provided at a second side of the joint opposite the first side. The method may further include determining a difference in the identified cross-sectional area of the first osteophyte and the identified cross-sectional area of the second osteophyte. The linear relationship may include a linear relationship between the determined difference in the identified cross-sectional areas and the adjustment to the planned bone resection angle such that, the greater the determined difference in the identified cross-sectional areas, the greater the adjustment to the planned bone resection angle.

The method may further include determining whether a difference in the identified cross-sectional areas is greater than or equal to a predetermined difference threshold. If the determined difference in the identified cross-sectional area is not greater than or equal to the predetermined difference threshold, the method may include determining that the first osteophyte and the second osteophyte are symmetric. If the determined difference in the identified cross-sectional area is greater than the predetermined difference threshold, the method may include determining that the first osteophyte and the second osteophyte are asymmetric.

If the first osteophyte and the second osteophyte are determined to be symmetric, then executing the algorithm may include determining that the planned bone resection depth should be decreased by a predetermined amount of depth per a predetermined amount of the identified cross-sectional area of the first osteophyte and/or the second osteophyte, or determining that the planned thickness of the implant should be increased by the predetermined amount of depth per the predetermined amount of the identified cross-sectional area of the first osteophyte and/or the second osteophyte. If the first osteophyte and the second osteophyte are determined to be asymmetric, then executing the algorithm may include determining, based on the identified cross-sectional area of the first osteophyte and the identified cross-sectional area of the second osteophyte, whether the first osteophyte is larger than the second osteophyte. If the first osteophyte is determined to be larger than the second osteophyte, the method may include determining that the planned bone resection angle should be adjusted in a first direction or orientation by a predetermined amount of bone resection angle per a predetermined amount of difference between the identified cross-sectional area of the first osteophyte and the identified cross-sectional area of the second osteophyte. If the first osteophyte is determined not to be larger than the second osteophyte, the method may include determining that the second osteophyte is larger than the first osteophyte, and determining that the planned bone resection angle should be adjusted in a second direction or orientation opposite the first direction or orientation by the predetermined amount of bone resection angle per the predetermined amount of difference.

The method may further include determining that the first osteophyte and the second osteophyte are asymmetric, determining that both the identified cross-sectional area of the first osteophyte and the identified cross-sectional area of the second osteophyte are greater than a predetermined cross-sectional area, determining that the difference in the identified cross-sectional areas is greater than a predetermined difference, determining that the planned bone resection depth should be decreased by the predetermined amount of depth per the predetermined amount of the identified cross-sectional area of the first osteophyte and/or the second osteophyte, or determining that the planned thickness of the implant should be increased by the predetermined amount of depth per the predetermined amount of the identified cross-sectional area of the first osteophyte and/or the second osteophyte.

The joint may be a knee joint. The first osteophyte may be a lateral osteophyte and the second osteophyte may be a medial osteophyte. The one or more bone cuts may include a tibial bone cut or a femoral bone cut. The first direction may be a tibial varus or a femoral varus. The second direction may be a tibial valgus or a femoral valgus. The predetermined amount of depth may be in a range of 0.4 millimeters (mm) to 0.6 mm. The predetermined amount of the identified cross-sectional area of the first osteophyte and/or the second osteophyte may be in a range of 85 mm2 to 100 mm2. The predetermined amount of bone resection angle may be in a range of 0.4° to 0.6°. The predetermined amount of difference between the identified cross-sectional area of the first osteophyte and the identified cross-sectional area of the second osteophyte may be in a range of 80 mm2 to 100 mm2.

The predetermined amount of depth may be in a range of 0.75 mm-0.125 mm. The predetermined amount of the identified cross-sectional area of the first osteophyte and/or the second osteophyte may be in a range of 15 mm² to 25 mm². The predetermined amount of bone resection angle may be in a range of 0.75°-0.125°. The predetermined amount of difference between the identified cross-sectional area of the first osteophyte and the identified cross-sectional area of the second osteophyte may be in a range of 15 mm² to 25 mm².

According to another aspect of the present disclosure, a method of assessing a joint may include receiving an image of a joint including an osteophyte and receiving a procedure plan including a plan to remove the osteophyte after one or more bone cuts are made. The image may show a view of the joint in a first dimension and a second dimension over which soft tissue extends over the osteophyte. The one or more bone cuts may be configured for installation of an implant during the procedure. The method may include identifying a cross-sectional area of the osteophyte in the first dimension and the second dimension and executing an algorithm to determine one or more adjustment parameters. The algorithm may apply a linear equation that receives, as input, the identified cross-sectional area of the at least one osteophyte, and outputs the one or more adjustment parameters. The one or more adjustment parameters may include an adjustment to one or more bone resection parameters and/or an adjustment to an implant parameter of the received procedure plan. The method may include outputting the one or more determined adjustment parameters to a display. The one or more bone resection parameters may include a planned bone resection depth of the one or more bone cuts and/or a planned bone resection angle of the one or more bone cuts.

According to yet another aspect of the present disclosure, a system configured to assess a joint may include an image acquisition device configured to acquire at least one image of the joint, a memory configured to store information, a controller, and a display. The information may include imaging data related to the at least on acquired image. The imaging data may include a cross-sectional area and position of at least one identified portion of a bone of the joint. The controller may be configured to execute an algorithm to determine, based on the at least one acquired image and/or the stored imaging data, one or more adjustment parameters. The algorithm may apply a linear equation that receives, as input, the cross-sectional area of the at least one identified portion of the bone, and outputs the one or more adjustment parameters. The one or more adjustment parameters may include an adjustment to a bone resection parameter and/or an adjustment to an implant parameter. The display may be configured to display the determined one or more adjustment parameters.

The image acquisition device may be a computed tomography (CT) acquisition device. The acquired at least one image may be a CT scan.

The CT scan may show a view of the joint in a first dimension and a second dimension over which the soft tissue extends. The cross-sectional area may be determined using the dimension of the identified portion of the bone in the first dimension and the second dimension.

The display may be configured to display a graphical user interface that includes a notification based on at least one identified portion of the bone identified in the acquired image.

The one or more adjustment parameters may include a coronal plane alignment, and the bone resection parameter may be used to determine the coronal plane alignment.

In yet another aspect of the present disclosure, a method of assessing a joint may include receiving an image of a joint, executing an algorithm to determine one or more adjustment parameters based on identification of a bony protrusion, such as a posteromedial bony flare, and outputting the one or more determined adjustment parameters to a display. The one or more adjustment parameters include a predicted change in soft tissue laxity after the identified bony protrusion is removed, an adjustment to a planned bone resection depth of the one or more bone cuts, an adjustment to a planned bone resection angle of the one or more bone cuts, and/or an adjustment to a planned size of the implant.

### BRIEF DESCRIPTION OF THE DRAWINGS

A more complete appreciation of the subject matter of this disclosure and the various advantages thereof may be understood by reference to the following detailed description, in which reference is made to the following accompanying drawings:

FIG. 1 illustrates an exemplary portion of a patient's anatomy, according to aspects of this disclosure.

FIG. 2 illustrates an exemplary view of soft tissue, such as a ligament, extending over a bone growth, such as an osteophyte, according to aspects of this disclosure.

FIG. 3 illustrates an exemplary view of the soft tissue of FIG. 2 with the osteophyte removed, according to aspects of this disclosure.

FIG. 4 illustrates an exemplary knee joint in flexion and extension, according to aspects of this disclosure.

FIG. 5 illustrates a cross-sectional view of an exemplary knee joint including one or more implant components, according to aspects of this disclosure.

FIG. 6 illustrates an exemplary electronic data processing system including a bone balancing system, according to an exemplary embodiment.

FIG. 7 illustrates exemplary process flow diagram including data, inputs, and outputs of the bone balancing system of FIG. 6, according to aspects of this disclosure.

FIG. 8 illustrates an exemplary graphical user interface of the bone balancing system of FIG. 6, according to aspects of this disclosure.

FIG. 9 illustrates an image of a patient's anatomy including a femur, a tibia, and an osteophyte, according to aspects of this disclosure.

FIG. 10 illustrates an example of detection and calculation of surface area of an osteophyte shown in FIG. 1, according to aspects of this disclosure.

FIG. 11 illustrates an example of detection and calculation of surface area of an osteophyte shown in FIG. 9, according to aspects of this disclosure.

FIG. 12 illustrates an exemplary preoperative method for adjusting a preoperative plan or procedure plan based on detected osteophytes, according to aspects of this disclosure.

FIG. 13 illustrates an exemplary preoperative and/or intraoperative method 1300 for adjusting a procedure plan based on detected osteophytes, according to aspects of this disclosure.

FIG. 14 illustrates an exemplary preoperative and/or intraoperative method for adjusting a procedure plan for a knee surgery based on detected osteophytes, according to aspects of this disclosure.

FIG. 15 illustrates an exemplary method for adjusting a surgical plan or procedure plan based on detected osteophytes, according to aspects of this disclosure.

FIG. 16 illustrates an exemplary method for adjusting a surgical plan or procedure plan based on laxity, according to aspects of this disclosure.

FIG. 17 illustrates example relationship of posterior osteophyte removal and laxity of a knee joint.

FIGS. 18A-D illustrate a superior view of a tibia and implant, overlaid with an exemplary guidance display in different stages of a surgical method, according to aspects of this disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “implant trial” and “trial” will be used interchangeably and as such, unless otherwise stated, the explicit use of either term is inclusive of the other term. In this disclosure, “user” is synonymous with “practitioner” and may be any person completing the described action (e.g., surgeon, technician, nurse, etc.).

An implant may be a device that is at least partially implanted in a patient and/or provided inside of a patient's body. For example, an implant may be a sensor, artificial bone, or other medical device coupled to, implanted in, or at least partially implanted in a bone, skin, tissue, organs, etc. A prosthesis or prosthetic may be a device configured to assist or replace a limb, bone, skin, tissue, etc., or portion thereof. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense.

In describing preferred embodiments of the disclosure, reference will be made to directional nomenclature used in describing the human body. It is noted that this nomenclature is used only for convenience and that it is not intended to be limiting with respect to the scope of the invention. For example, as used herein, the term “distal” means toward the human body and/or away from the operator, and the term “proximal” means away from the human body and/or towards the operator. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

The present disclosure generally provides for systems and methods of detection and resection of osteophytes, as well as balancing joint laxity of a knee joint. Osteophytes may develop around the knee in response to disease, and osteophyte number, size, volume, and growth may correlate to disease progression. As osteophytes grow, soft tissues extending over the osteophytes may be stretched. However, once osteophytes are removed, the previously stretched soft tissues may be slack and may not fully contract to an initial state. This reduced laxity in soft tissue may result in the joint laxity. Thus, for gap balancing, a surgeon may try to predict the effect of osteophyte removal on the resulting flexion and extension gap before any bone cuts are made and before those osteophytes are removed.

In some examples, with conventional Total Knee Arthroplasty (TKA), the surgeon may adjust the soft tissue to the bone cuts. In some examples with a robotic TKA, the surgeon may adjust the bone cuts to the soft tissue. In some examples, debridement of osteophytes may require adjustment to the planned bone resection. To better plan a TKA with target laxity, a predictive algorithm to determine the precise adjustment in bone resection required based on osteophyte size and location may be desired and is discussed in further detail hereinbelow.

Osteophytes may be found in many locations: medial, lateral, anterior, posterior, central within the intercondylar notch, and peripatellar. Most osteophytes may be readily accessible and can be removed during the surgical approach to the knee. Posterior osteophytes, however, are relatively inaccessible, in the sense that their removal may only be done after posterior bone cuts are made on the femur. Any laxity created by posterior osteophyte removal cannot be corrected by adjusting bone cuts because the bone cuts have already been made. The present disclosure generally provides for a predictive algorithm which anticipates the effect of osteophyte removal before the osteophytes are removed, allowing adjustment in bony resection before any bone cuts are made.

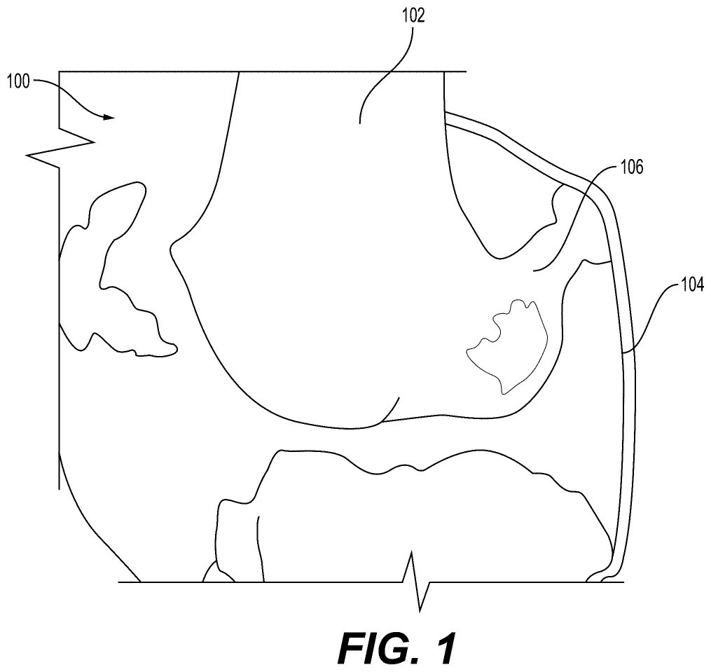

FIG. 1 illustrates an exemplary portion of anatomy 100 (e.g., leg or knee joint). Referring to FIG. 1, the portion of anatomy 100 may include one or more bones 102 (e.g., tibia or femur) and one or more soft tissues 104 (e.g., ligament). The one or more bones 102 may include one or more osteophytes 106 or other bony protrusions. The soft tissue 104 may extend over the osteophyte 106.

An osteophyte may be a bone spur that develops on a bone. Osteophytes may develop in response to disease (e.g., osteoarthritis or OA), and osteophyte number, size, volume, and/or growth may correlate to disease progression. As osteophytes grow, soft tissues extending over the osteophytes may be stretched. With reference to FIG. 1, the soft tissue 104 (e.g., ligament) may be stretched over the osteophyte 102.

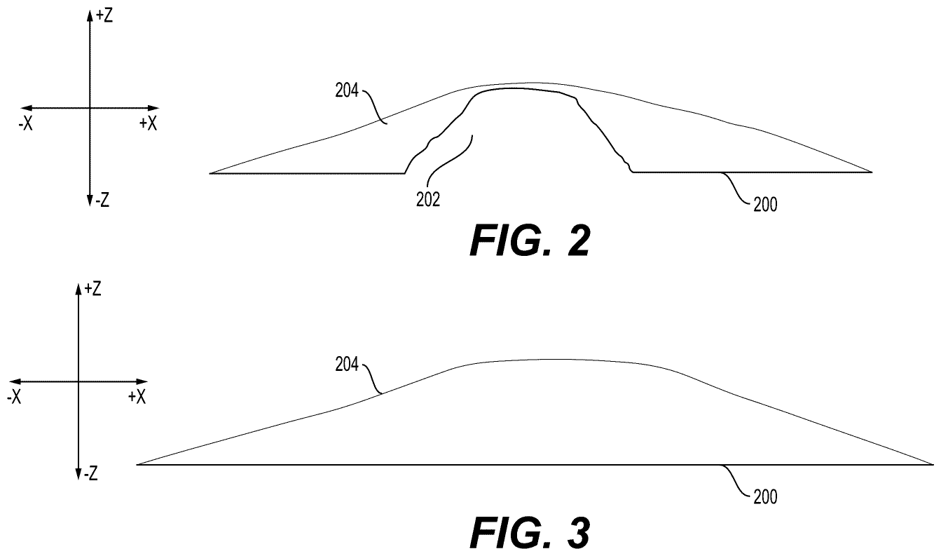

Removing osteophytes during a medical procedure (e.g., total knee arthroplasty or TKA, or partial knee arthroplasty or PKA) may be desired to treat disease and also reduce pain (e.g., from impingement). However, once removed, the previously stretched soft tissues may be slack and may not fully contract to an initial state due to the removal of the one or more osteophytes. For example, referring to FIG. 2, a bone 200 may include an osteophyte 202 and a ligament 204 extending over the osteophyte 202. Referring to FIG. 3, when the osteophyte 202 is removed from the bone 200, the ligament 204 may remain slack. If the ligament and osteophyte 202 occur near a joint (e.g., knee joint), this reduced laxity in soft tissue may result in the joint being looser, potentially reducing the functionality of the joint and increasing patient complications.

As shown in FIG. 2, the ligament 204 may extend along an X-direction and a Z-direction over the osteophyte 202, and may therefore be stretched out in the X-direction and Z-direction when the osteophyte 202 is removed (FIG. 3). A thickness or width of the osteophyte 202 in a direction into and/or out of the page, however, may not be relevant to how the ligament 204 in FIGS. 2 and 3 is stretched.

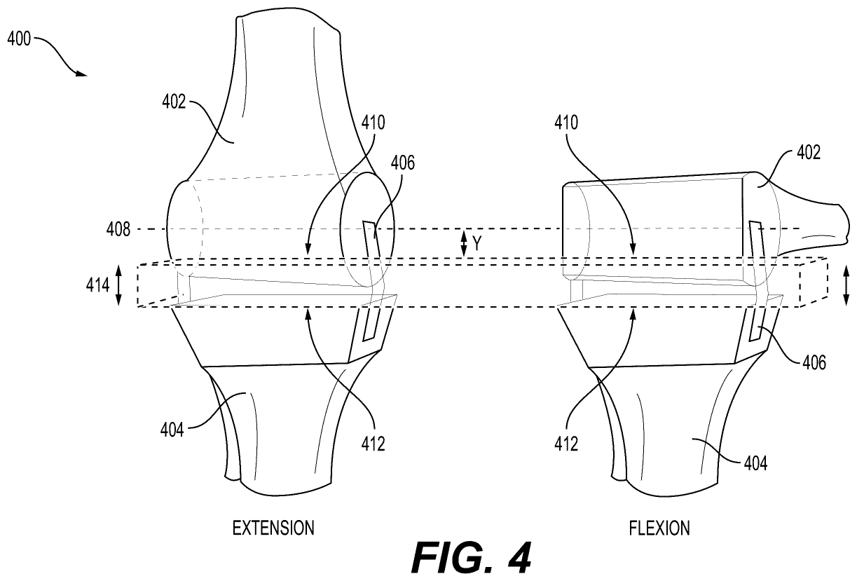

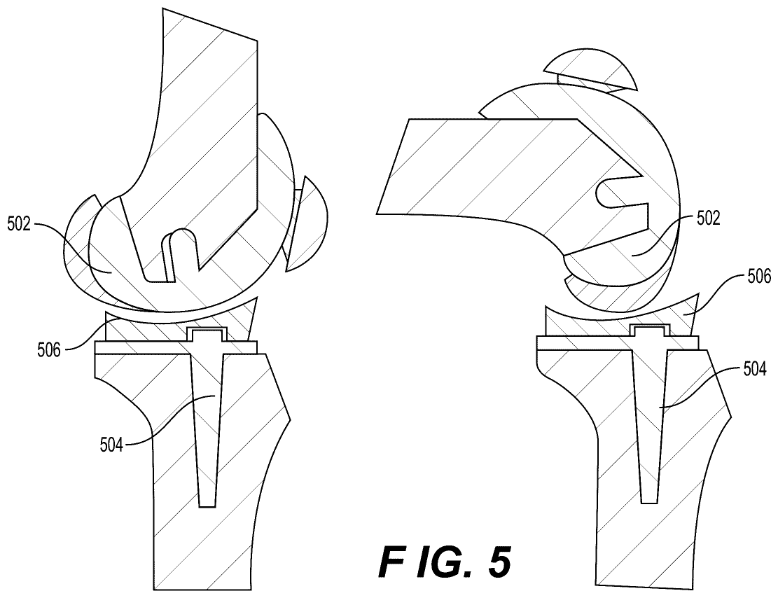

FIG. 4 illustrates an exemplary knee joint 400 in flexion and extension. Referring to FIG. 4, the knee joint 400 may include a femur 402, a tibia 404, and a ligament 406 (e.g. medial collateral ligament) coupled to the femur 402 and the tibia 404. The femur 402 may rotate with respect to tibia 404 about a flexion-extension axis 408 extending through the femur 402. A procedure plan (e.g, surgery plan or medical operation plan) may include one or more bone cuts, such as a distal and posterior femoral resection 410 through the femur 402 and/or a proximal tibial resection 412 through the tibia 404. When the knee joint 400 is in extension, an extension gap 414 may extend between the femoral resection 410 and the tibial resection 412. When the knee joint 400 is in flexion, a flexion gap 416 may extend between the femoral resection 410 and the tibial resection 412. The femoral resection 410 and the tibial resection 412 may be configured to provide a balanced knee joint 400, and in some examples, in conjunction with one or more prosthetic components or liners such as the femoral prosthetic component 502, the tibial prosthetic component 504, and the liner 506 shown in FIG. 5.

When planning for a medical procedure at the knee joint 400, a practitioner (e.g., surgeon, doctor, healthcare planner, etc.) may desire to make the extension gap 414 equal to the flexion gap 416. In some examples, the practitioner may plan to create a rectangular flexion gap 416 by adjusting a femoral cutting block so that the femoral cutting block is parallel to a resected tibial surface at 90° with the ligaments (e.g., ligament 406) under tension. These adjustments and the laxity of the ligament 406, as explained above, are effected by the presence of and later removal of osteophytes. Thus, the practitioner may remove any readily accessible osteophytes prior to planning (or fine tuning of the plan) and making bone cuts, but certain osteophytes (e.g., posterior osteophytes) may not be accessible until after bone cuts are made. The later removal of the posterior osteophytes may significantly affect the extension gap 414 and the flexion gap 416. Thus, for gap balancing, a practitioner may try to predict the effect of posterior osteophyte removal on the resulting extension gap 414 and the flexion gap 416 before any bone cuts are made and before those posterior osteophytes are removed, similar to how a golfer may make an adjustment in aim to compensate for prevailing wind. Removal of a posterior osteophyte, whether medial or lateral in location, may effect both extension and flexion.

Aspects disclosed herein may analyze a position and/or dimension of these initially inaccessible osteophytes to determine one or more bone resection parameters (e.g., bone cut position and/or slope) for a procedure. Alternatively or in addition thereto, aspects disclosed herein may analyze a position and/or dimension of these initially inaccessible osteophytes to determine a dimension and/or design of one or more implants or prosthetics used in a procedure, such a thickness, size, material, and/or shape of the femoral prosthetic component 502, the tibial prosthetic component 504, and/or the liner 506 shown in FIG. 5.

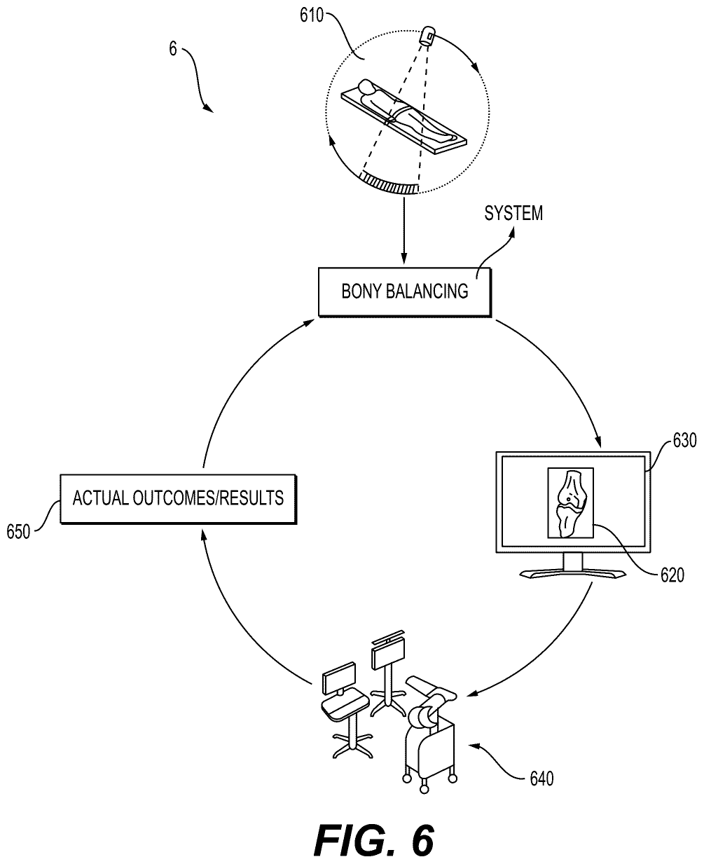

Referring to FIG. 6, an electronic data processing system 6 may include a bone balancing system 600 including one or more algorithms. The bone balancing system 600 may receive one or more medical images of patient's anatomy acquired using one or more image acquisition devices 610, analyze the received one or more medical images to determine and/or adjust a procedure plan 620, which may be output into a display 630 and/or to a robotic and/or automated data system or platform 640 (e.g., a robotic system such as a surgical robot and/or a robotic tool). Results and/or outcome data 650 from the procedure may be fed to the bone balancing system 600 for further refinement of the one or more algorithms.

The bone balancing system 600 may be implemented as one or more computer systems or cloud-based electronic processing systems. The image acquisition device 610 may include a computed tomography (CT) scanner, a magnetic resonance imaging (MRI) machine, an x-ray machine, a radiography system, an ultrasound system, a thermography system, a tactile imaging system, an electrography, nuclear medicine functional imaging system, a positron emission tomography (PET) system, a single-photon emission computer tomography (SPECT) system, a camera, etc. An instant patient who is planning to undergo a procedure (e.g., surgery) may first undergo imaging using the image acquisition device 610 (e.g., a CT scanner). Images and/or information collected during imaging (e.g., CT or CAT scans) may be transmitted from or stored in the image acquisition device 610.

During imaging using the image acquisition device 610, the patient may undergo one or more “poses” or positions where certain soft tissues of a joint (e.g., medial and/or lateral soft tissues) are stressed in certain positions of the joint (e.g., flexion and extension) by applying one or more forces (e.g., varus and valgus forces). The bone balancing system 600 may determine and/or calculate a size and/or shape of one or more gaps of the joint (e.g., flexion and extension gaps) to assess the soft tissue envelope of the joint. Alternatively or in addition thereto, the practitioner may determine a size and/or shape of the one or more gaps, and input the determined size and/or shape into the bone balancing system 600 (e.g., via a user interface such as display 630). The bone balancing system 600 may identify bone landmarks from the images and/or data (e.g., osteophytes and their dimensions and/or positions) collected at the poses. Alternatively, a practitioner may analyze the images and input, into the bone balancing system 600, imaging data, such as positions and dimensions of bone landmarks (e.g., osteophytes).

The bone balancing system 600 may execute the one or more algorithms, using the imaging data including the determined gap (e.g., flexion and extension gap) and/or the identified bone landmarks (e.g., osteophytes) to determine the procedure plan 620. The procedure plan 620 may include a series of steps to perform in the procedure, such as one or more bone resection parameters (e.g., resections or cuts to make in a bone and/or one or more osteophytes to remove) and/or a design of an implant (e.g., prosthetic liner). The procedure plan 620 may also include predicted outcomes (e.g., a risk of complication during the procedure or a risk of infection post-procedure). As an example, the procedure plan 620 may first be determined based on the size and/or the shape of the one or more gaps of the joint, and then may be revised based on identified osteophytes that would be planned to be removed during the procedure.

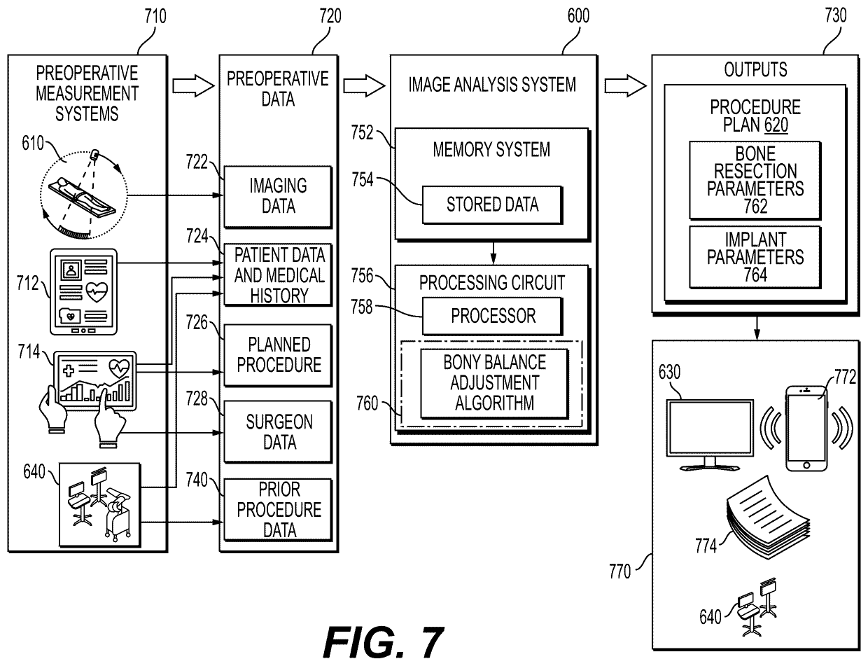

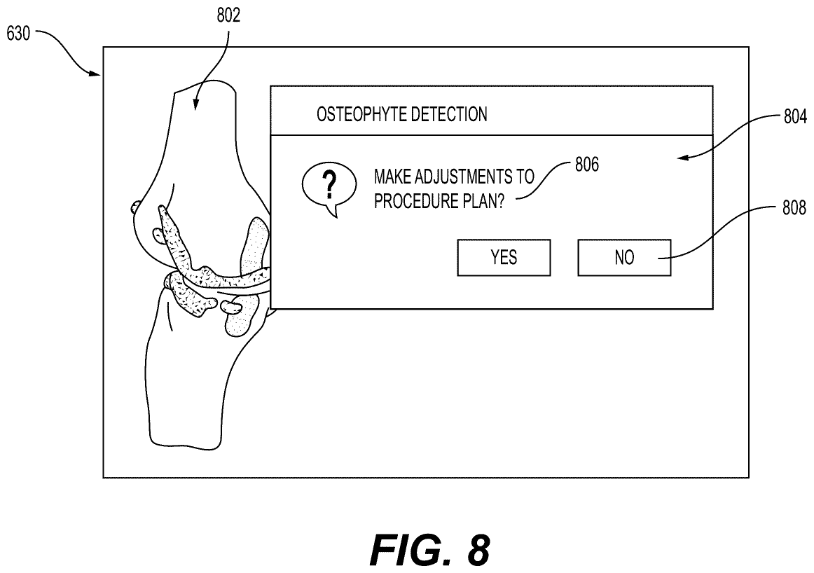

In some examples, the procedure plan 620, including the one or more bone resection parameters 762 and/or implant parameters 764 (FIG. 7), may be transmitted to a display 630. The one or more bone parameters 762 and the one or more implant parameters 764 of the procedure plan 620 may collectively be referred to as one or more adjustment parameters. In some examples, the procedure plan 620, including the one or more bone resection parameters 762 and/or implant parameters 764, may be transmitted to a surgical robot or robotic tool (e.g., an automated cutting burr) of the robotic and/or automated data system 640 to execute the determined bone resection parameters 762 by, for example, automatically cutting, via a surgical robot holding a tool, a surgeon holding a robotic tool, etc., a bone according to the bone resection parameters 762 of the procedure plan 620. As the course of treatment is continued, the actual or observed outcomes and/or results 650 may also be used by the bone balancing system 600 to either update its predictions (e.g., intraoperatively based on intraoperatively collected data or outcomes) and/or to make future predictions for future patients (e.g., based on a postoperative result or outcome). Intraoperative data for further refinements may be similar to the preoperative data 720. Details of the bone balancing system 600 and its determinations will be described in more detail with respect to FIG. 7.

FIG. 7 illustrates exemplary data, inputs, and outputs of the bone balancing system 600. Referring to FIG. 7, the bone balancing system 600 may receive preoperative data 720 from one or more preoperative measurement systems 710 and analyze the preoperative data 720 to produce one or more outputs 730 such as the procedure plan 620 to one or more output systems 770, such as the display 630. The preoperative measurement systems 710 may include the image acquisition device 610; electronic devices storing electronic medical records (EMR) 714; patient, practitioner, and/or user interfaces or applications 750 (such as on tablets, computers, or other mobile devices); and the robotic and/or automated data system or platform 640 (e.g., MAKO Robot System or platform, MakoSuite, etc.), which may include a robotic device, as previously mentioned.

The bone balancing system 600 may receive imaging data 722 via the image acquisition device 610 and supplemental or additional information (e.g., patient data and medical history 724, planned procedure data 726, surgeon and/or staff data 728, and/or prior procedure data 740) via EMR 712, interfaces 714, sensors and/or electronic medical devices, and/or robotic platform 640. Each of the devices in the preoperative measurement systems 710 (the image acquisition device 610, EMR 712, user interfaces or applications 714, sensors and/or electronic medical devices, and robotic platform 640) may include one or more communication modules (e.g., WiFi modules, BlueTooth modules, etc.) configured to transmit preoperative data 720 to each other, to the bone balancing system 600, and/or to the one or more output systems 770.

The image acquisition device 610 may be configured to collect or acquire one or more images, videos, or scans of a patient's internal anatomy, such as bones, ligaments, soft tissues, brain tissue, etc. to provide imaging data 722, which will be described in more detail later. As previously described, the image acquisition device 610 may include a computed tomography (CT) scanner, a magnetic resonance imaging (MRI) machine, an x-ray machine, a radiography system, an ultrasound system, a thermography system, a tactile imaging system, an electrography, nuclear medicine functional imaging system, a positron emission tomography (PET) system, a single-photon emission computer tomography (SPECT) system, a camera, etc. The collected images, videos, or scans may be transmitted, automatically or manually, to the bone balancing system 600. In some examples, a user may select specific images from a plurality of images taken with the image acquisition device 610 to be transmitted to the bone balancing system 600.

The bone balancing system 600 may use previously collected data from EMR 712, which may include patient data and medical history 724 in the form of past practitioner assessments, medical records, past patient reported data, past imaging procedures, treatments, etc. For example, EMR 712 may contain data on demographics, medical history, biometrics, past procedures, general observations about the patient (e.g., mental health), lifestyle information, data from physical therapy, etc.

The bone balancing system 600 may also use present or current (e.g., in real time) patient data via patient, practitioner, and/or user interfaces or applications 714. These user interfaces 714 may be implemented on mobile applications and/or patient management websites or interfaces, such as OrthologIQ®. User interfaces 714 may present questionnaires, surveys, or other prompts for practitioners or patients to enter assessments (e.g., throughout a prehabilitation program prior to a procedure), observed data and/or reactions (e.g., in response to various poses), psychosocial information and/or readiness for surgery, comments, etc. for additional patient data 724. Patients may also enter psychosocial information such as perceived or evaluated pain, stress level, anxiety level, feelings, and other patient reported outcome measures (PROMS) into these user interfaces 714. Patients and/or practitioners may report lifestyle information via user interfaces 714. User interfaces 714 may also collect clinical data such as planned procedure 726 data and planned surgeon and/or staff data 728 described in more detail later. These user interfaces 714 may be executed on and/or combined with other devices disclosed herein (e.g., with robotic platform 640).

The bone balancing system 600 may receive prior procedure data 750 from prior patients and/or other real-time data or observations (e.g., observed patient data 724) via robotic platform 640. The robotic platform 640 may include one or more robotic devices (e.g., surgical robot), computers, databases, etc. used in prior procedures with different patients. The robotic platform 640 may have assisted with, via automated movement, surgeon assisted movement, and/or sensing, a prior procedure and may be implemented as or include one or more automated or robotic surgical tools, robotic surgical or Computerized Numerical Control (CNC) robots, surgical haptic robots, surgical tele-operative robots, surgical hand-held robots, or any other surgical robot.

Although the preoperative measurement system(s) 710 is described in connection with image acquisition device 610, EMR 712, user interfaces 714, and robotic platform 640, other devices may be used preoperatively to collect preoperative data 720, for example data relating to joint alignment and/or identified bone landmarks or osteophytes or other data sued to create procedure plan 620. For example, mobile devices such as cell phones and/or smart watches may include various sensors (e.g., gyroscopes, accelerometers, temperature sensors, optical or light sensors, magnetometer, compass, global positioning systems (GPS) etc.) to collect patient data 724 such as location data, sleep patterns, movement data, heart rate data, lifestyle data, activity data, etc. As another example, wearable sensors, heart rate monitors, motion sensors, external cameras, etc. having various sensors (e.g., cameras, optical light sensors, barometers, GPS, accelerometers, temperature sensors, pressure sensors, magnetometer or compass, MEMs devices, inclinometers, acoustical ranging, etc.) may be used during physical therapy or a prehabilitation program to collect information on patient kinematics, alignment, movement, fitness, heart rate, electrocardiogram data, breathing rate, temperature, oxygenation, sleep patterns, activity frequency and intensity, sweat, perspiration, air circulation, stress, step pressure or push-off power, balance, heel strike, gait, fall risk, frailty, overall function, etc. Other types of systems or devices may include electromyography or EMG systems or devices, motion capture (mocap) systems, sensors using machine vision (MV) technology, virtual reality (VR) or augmented reality (AR) systems, etc.

The preoperative data 720 may be data collected, received, and/or stored prior to an initiation of a medical treatment plan or medical procedure. As shown by the arrows in FIG. 7, the preoperative data 720 may be collected using the preoperative measurement systems 710, from a memory system 752 (e.g., cloud storage system) of the bone balancing system 600, and from output systems 770 (e.g., from a prior procedure) for one or more continuous feedback loops. Some of the preoperative data 724 may be directly sensed via one or more devices (e.g., image acquisition device 610 and/or wearable motion sensors or mobile devices) or may be manually entered by a medical professional, patient, or other party. Other preoperative data 724 may be determined (e.g., by bone balancing system 600) based on directly sensed information, input information, and/or stored information from prior medical procedures.

As previously described, the preoperative data 724 may include imaging data 722, patient data and/or medical history 724, information on a planned procedure 726, surgeon data 728, and prior procedure data 740.

The imaging data 722 may include one or more images (e.g., raw images), videos, or scans of a patient's anatomy collected and/or acquired by the image acquisition device 610. The bone balancing system 600 may receive and analyze one or more of these images to determine further imaging data 722, which may be used as further input preoperative data 724. In some example, image acquisition device 610 may analyze and/or process the one or more images, and send any analyzed and/or processed imaging data to the bone balancing system 600 for further analysis.

The one or more images of the imaging data 722 may illustrate or indicate, and the bone balancing system 600 may be configured to identify and/or recognize in the images: bone, osteophyte, soft tissue, or cartilage positions or alignment, composition and/or density, fractures and/or tears, bone landmarks (e.g., condyle surface, head or epiphysis, neck or metaphysis, body or diaphysis, articular surface, epicondyle, lateral epicondyle, medial epicondyle, process, protuberance, tubercle vs tuberosity, tibial tubercle, trochanter, spine, linea or line, facet, crests and ridges, foramen and fissure, meatus, fossa and fovea, incisure and sulcus, and sinus), geometry (e.g., diameters, slopes, angles) and/or other anatomical geometry data such as deformities or flare (e.g., coronal plane deformity, sagittal plane deformity, lateral femoral metaphyseal flare, or medial femoral metaphysical flare). Such geometry is not limited to overall geometry and may include relative dimensions (e.g., lengths or thicknesses of a tibia or femur).

The imaging data 722 may include information about identified or detected osteophytes at or around a joint, such as a knee joint. The imaging data 722 may include dimensions and/or positioning of the osteophytes, a compartment or location of the osteophytes, and/or an indication of whether the osteophytes are readily accessible or can only be removed after bone cuts. The imaging data 722 may indicate or be used to determine osteophyte size, volume, or positions; bone loss; joint space; B-score; bone quality/density; skin-to-bone ratio; bone loss; hardware detection; anterior-posterior (AP) and medial-lateral (ML) distal femur size, and/or joint angles. Analysis and/or calculations that may be derived from the images or scans will be described in more detail later when describing the bone balancing system 600. For example, autonomous segmentation and/or calculation of osteophyte size and location may be applied to the one or more images and/or imaging data 722.

In addition, the imaging data 722 may include morphology and/or anthropometrics (e.g., physical dimensions of internal organs, bones, etc.), fractures, slope or angular data, tibial slope, posterior tibial slope or PTS, bone density, (e.g., bone mineral or bone marrow density, bone softness or hardness, or bone impact), etc. Imaging data 722 may not be limited to bone data and may be inclusive of other internal imaging data, such as of cartilage, soft tissue, blood flow, or ligaments.

In addition to raw images, imaging data 722 may include intermediate and/or related imaging data 722 to be used by the bone balancing system 600 to calculate outputs 730. Such intermediate imaging data 722 may include density or composition charts or graphs; quantified data indicating relative positions, dimensions, etc.; and/or processed image data indicating specifically detected attributes, such as a probability of a certain patient condition. One or more algorithms 760 of the bone balancing system 600 may determine or calculate this intermediate imaging data 722 in determining outputs 730, or alternatively or additionally thereto, the image acquisition device 610 may include one or more processors configured to calculate or quantify, based on the raw images, videos, or scans, at least some of the intermediate imaging data 722. Intermediate imaging data 722 may include information relating to, indicating, and/or quantifying aspects of the raw images, charts, etc.

Patient data and medical history 724 may include information about the instant patient on identity (e.g., name or birthdate), demographics (e.g., patient age, gender, height, weight, nationality, body mass index (BMI), etc.), lifestyle (e.g., smoking habits, exercise habits, drinking habits, eating habits, fitness, activity level, frequency of climbing activities such as up and down stairs, frequency of sit-to-stand movements or bending movements such as when entering and exiting a vehicle, steps per day, activities of daily living or ADLs performed, etc.), medical history (e.g., allergies, disease progressions, addictions, prior medication use, prior drug use, prior infections, frailties, comorbidities, prior surgeries or treatment, prior injuries, prior pregnancies, utilization of orthotics, braces, prosthetics, or other medical devices, etc.), assessments and/or evaluations (e.g., laboratory tests and/or bloodwork, American Society of Anesthesiology or ASA score and/or fitness for surgery or aesthesia) electromyography data (muscle response or electrical activity in response to a nerve's stimulation), psychosocial information (e.g., perceived pain, stress level, anxiety level, mental health status, PROMS (e.g., knee injury and osteoarthritis outcome score or KOOS, hip disability and osteoarthritis outcome score or HOOS, pain virtual analog scale or VAS, PROMIS Global 10 or PROMIS-10, EQ-5D, a mental component summary, satisfaction or expectation information, etc.), past biometrics (e.g., heart rate or heat rate variability, electrocardiogram data, breathing rate, temperature (e.g., internal or skin temperature), fingerprints, DNA, etc.), past kinematics or alignment data, past imaging data, data from prehabilitation programs or physical therapy (e.g., average load bearing time) etc. Medical history 724 may include prior clinical or hospital visit information, including encounter types, dates of admission, hospital-reported comorbidity data such as Elixhauser and/or Charlson scores or selected comorbidities (e.g., ICD-10 POA), prior anesthesia taken and/or reactions, etc. This list, however, is not exhaustive and preoperative data 720 may include other patient specific information, clinical information, and/or surgeon or practitioner specific information (e.g., experience level).

Patient data 724 may come from EMR 712, user interfaces 714, from memory system 752, and/or from robotic platform 640, but aspects disclosed herein are not limited to a collection of the patient data 724. For example, other types of patient data 724 or additional data may include data on activity level; kinematics; muscle function or capability; range of motion data; strength measurements and/or force measurements push-off power, force, or acceleration; a power, force, or acceleration at a toe during walking; angular range or axes of joint motion or joint range of motion; flexion or extension data, including step data (e.g., measured by a pedometer), gait data or assessments; fall risk data; balancing data; joint stiffness or laxity data; postural sway data; data from tests conducted in a clinic or remotely; etc.

Information on a planned procedure 726 may include an initial procedure plan 620 and/or logistical information about the procedure and substantive information about the procedure. Substantive planned procedure 726 information may include a surgeon's surgical or other procedure or treatment plan, including planned steps or instructions on incisions, a side of the patient's body to operate on (e.g., left or right) and/or laterality information, bone cuts or resection depths, implant design, type, and/or size, implant alignment, fixation or tool information (e.g., implants, rods, plates, screws, wires, nails, bearings used), cementing versus cementless techniques or implants, final or desired alignment, pose or orientation information (e.g., capture gap values for flexion or extension, gap space or width between two or more bones, joint alignment), planning time, gap balancing time, extended haptic boundary usage, etc. Logistical planned procedure 726 information may include information about a planned site of the procedure such as a hospital, a type of procedure or surgery to be performed (e.g., total or partial knee arthroplasty or replacement, total or partial hip arthroplasty or replacement, spine surgery, patella resurfacing), scheduling or booking information, equipment or tools required, etc. This initial planned procedure 726 information may be manually prepared or input by a surgeon and/or previously prepared or determined using one or more algorithms. Surgeon data 728 may include information about a surgeon or other staff planned to perform the procedure plan 620 such as identity (e.g., name), experience level, fitness level, height and/or weight, etc.

Prior procedure data 740 may include information about prior procedures performed on a same or prior patient. Such information may include the same type of information as in planned procedure data 728 (e.g., instructions or steps of a procedure, bone cuts, implant design, implant alignment, etc.) along with outcome and/or result information (e.g., outcomes 650 described with reference to FIG. 6), which may include both immediate results and long-term results, complications after surgery, length of stay in a hospital, revision surgery data, rehabilitation data, patient motion and/or movement data, etc. Prior procedure data 740 may include information about prior procedures of prior patients sharing at least one same or similar characteristic (e.g., demographically, biometrically, disease state, etc.) as the instant patient.

Preoperative data 720 may include any other additional or supplemental information stored in memory system 752, which may also include known data and/or data from third parties, such as data from the Knee Society Clinical Rating System (KSS) or data from the Western Ontario and McMaster Universities Osteoarthritis Index (WOMAC). In addition, in some examples, intraoperative may be collected and/or received by the bone balancing system 600 to further generate and/or refine the outputs 730 (e.g., procedure plan 620).

### The Bone Balancing System 600

The bone balancing system 600 may include a memory system 752 including stored data 754 and a processing circuit 756 including a processor 758 and one or more algorithms. The one or more algorithms may include a bone balance adjustment algorithm 760. The bone balance adjustment algorithm 760 may use one or more linear relationships, non-linear relationships, or both to analyze the imaging data 722 to generate and/or modify the procedure plan 620. In some examples, the bone balancing system 600 may include an artificial intelligence (AI) and/or machine learning system that is “trained” or that may learn and refine the relationships, patterns, etc. between preoperative data 720, outputs 730, and actual results 650 (FIG. 6) to make determinations and/or, in some examples, to refine the bone balance adjustment algorithm 760. The bone balancing system 600 and the bone balance adjustment algorithm 760 may alternatively be referred to as a bony balancing system 600 and a bony balance adjustment algorithm 760 and/or a gap or soft tissue balancing system 600 or a gap or soft tissue balance adjustment algorithm 760.

The bone balancing system 600 may be implemented using one or more computing platforms, such as platforms including one or more computer systems and/or electronic cloud processing systems. Examples of one or more computing platforms may include, but are not limited to, smartphones, wearable devices, tablets, laptop computers, desktop computers, Internet of Things (IoT) device, remote server/cloud based computing devices, or other mobile or stationary devices. The bone balancing system 600 may also include one or more hosts or servers connected to a networked environment through wireless or wired connections. Remote platforms may be implemented in or function as base stations (which may also be referred to as Node Bs or evolved Node Bs (eNBs)). Remote platforms may also include web servers, mail servers, application servers, etc.

The bone balancing system 600 may include one or more communication modules (e.g., WiFi or Bluetooth modules) configured to communicate with preoperative measurement systems 722, output system 770, and/or other third-party devices, etc. For example, such communication modules may include an Ethernet card and/or port for sending and receiving data via an Ethernet-based communications link or network, or a Wi-Fi transceiver for communication via a wireless communications network. Such communication modules may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, Bluetooth, near field communication (NFC), radio frequency identifier (RFID), ultrawideband (UWB), etc.). Such communication modules may include a radio interface including filters, converters (for example, digital-to-analog converters and the like), mappers, a Fast Fourier Transform (FFT) module, and the like, to generate symbols for a transmission via one or more downlinks and to receive symbols (for example, via an uplink).

The memory system 752 may have one or more memories or storages configured to store or maintain the preoperative data 720, outputs 730, and stored data 754 from prior patients and/or prior procedures. The preoperative data 720 and outputs 730 of an instant procedure may also become stored data 754. Although the memory system 752 is illustrated close to processing circuit 756, memory system 752 may include memories or storages implemented on separate circuits, housings, devices, and/or computing platforms and in communication with bone balancing system 600, such as cloud storage systems and other remote electronic storage systems.

The memory system 752 may include one or more external or internal devices (random access memory or RAM, read only memory or ROM, Flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein. The memory system 20 may include volatile memory or non-volatile memory (e.g., semiconductor-based memory device, a magnetic memory device and system, an optical memory device and system, fixed memory, or removable memory). The memory system 752 may include database components, object code components, script components, or any other type of information structure to support the various activities described herein. In some aspects, the memory system 752 may be communicably connected to the processing circuit 756 and may include computer code to execute one or more processes described herein. The memory system 752 may contain a variety of modules, each capable of storing data and/or computer code related to specific types of functions.

The processing circuit 756 may include a processor 758 configured to execute or perform the one or more algorithms, including the bone balance adjustment algorithm 760, based on received data, which may include the preoperative data 720 and/or any data in the memory system 752 to determine the outputs 730. The preoperative data 720 may be received via manual input, retrieved from the memory system 752, and/or received direction from the preoperative measurement systems 722. The processor 758 may be configured to determine patterns based on the received data.

The processor 758 may be implemented as a general purpose processor or computer, special purpose computer or processor, microprocessor, digital signal processor (DSPs), an application specific integrated circuit (ASIC), one or more field programmable gate arrays (FPGAs), a group of processing components, processor based on a multi-core processor architecture, or other suitable electronic processing components. The processor 758 may be configured to perform machine readable instructions, which may include one or more modules implemented as one or more functional logic, hardware logic, electronic circuitry, software modules, etc. In some cases, the processor 758 may be remote from one or more of the computing platforms comprising the bone balancing system 600. The processor 758 may be configured to perform one or more functions associated with the bone balancing system 600, such as precoding of antenna gain/phase parameters, encoding and decoding of individual bits forming a communication message, formatting of information, and overall control of one or more computing platforms comprising the bone balancing system 600, including processes related to management of communication resources and/or communication modules.

In some aspects, the processing circuit 756 and/or memory system 752 may contain several modules related to medical procedures, such as an input module, an analysis module, and an output module. The bone balancing system 600 need not be contained in a single housing. Rather, components of the bone balancing system 600 may be located in various different locations or even in a remote location. Components of the bone balancing system 600, including components of the processing circuit 756 and the memory system 752, may be located, for example, in components of different computers, robotic systems, devices, etc. used in surgical procedures.

The bone balancing system 600 may use the one or more algorithms, including the bone balance adjustment algorithm 760, to make intermediate determinations and to determine the one or more outputs 730. The one or more algorithms may be configured to determine or glean data from the preoperative data 720, including the imaging data 722. For example, the one or more algorithms may be configured for bone recognition, soft tissue recognition, and/or to make determinations related to the intermediate imaging data 722 previously described. The one or more algorithms may operate simultaneously and/or separately to determine the one or more outputs 730 and/or display the one or more outputs 730.

The one or more algorithms include the bone balance adjustment algorithm 760. In addition, the one or more algorithms may include one or more machine learning algorithms that are trained using, for example, linear regression, non-linear regression, random forest regression, CatBoost regression, statistical shape modelling or SSM, etc. The one or more algorithms may be continuously modified and/or refined based on actual outcomes and/or results 650 (FIG. 6). The one or more algorithms may be configured to use segmentation techniques and/or thresholding techniques on received images, videos, and/or scans of the imaging data 722 to determine the one or more outputs 2722. For example, the one or more algorithms may be configured to segment an image (e.g., a CT scan, an ultra sound image) and/or threshold soft tissue or identified bone, bone landmarks, osteophytes, etc. The one or more algorithms may be configured to automate data extraction and/or collection upon receiving an image from the image acquisition device 610. The bone balance adjustment algorithm 760 will be described in more detail hereinafter.

The one or more outputs 730 may include the procedure plan 620, but are not limited thereto. For example, the one or more outputs 730 may include related graphics, texts, or graphical user interfaces (GUIs) to display the procedure plan 620, patient anatomy representations, determined and/or enhanced images, etc. configured to be displayed on the display 630 or other output systems 770. Each of these outputs 730 may be used as input preoperative data 722 to determine other outputs 730.

The outputs 730 may be output to any one or more of the output systems 770 via a wireless, electronic, and/or wired connection. The output systems 770 may include the display 630, a mobile device 772, or physical paper, canvas, film or other physical medium 774.

### The Bone Balance Adjustment Algorithm 760

To determine the one or more bone resection parameters 762 and/or implant parameters 764, the bone balancing system 600 may execute the bone balance adjustment algorithm 760. The bone balance adjustment algorithm 760 may be one algorithm and/or may include multiple algorithms that perform similar calculations (e.g., one algorithm for each type of bone cut, soft tissue envelope, etc.) and/or that perform related functions (e.g., bone recognition, surface area calculation, and bone parameter determination). For convenience of description, the bone balance adjustment algorithm 760 will be described as one algorithm. Although the bone balancing system 600 is described as executing the bone balance adjustment algorithm 760, aspects disclosed herein are not limited to a device or system that executes the bone balance adjustment algorithm 760.

As previously described, the bone balance adjustment algorithm 760 may be configured to determine the one or more bone resection parameters 762 and/or one or more implant parameters 764 for a joint based on identified osteophytes, identified location and/or dimensions of the osteophytes, and their planned removal. The bone resection parameters 762 may include a laxity parameter, a bone cut adjustment, a bone cut depth, a bone cut angle or slope, or other adjustments a surgeon may make during a procedure where bone resections are made. The implant parameters 764 may include one or more dimensions of an implant configured to balance the joint and/or a gap of the joint, such as an implant thickness.

For example, the bone balance adjustment algorithm 760 may be configured to predict an amount of soft tissue laxity that will be created by removing identified osteophytes based on a size and/or location of the osteophyte before they are removed. For example, when posterior osteophytes are present on a femur or a tibia, a practitioner may plan to remove them. Due to their posterior location, access to posterior osteophytes may only be possible after tibial and femoral bone cuts are made. Once the osteophytes are removed, the soft tissue envelope may change, effectively changing both the size and shape of flexion and extension gaps, rendering the pre-resection soft tissue “poses” inaccurate. Thus, the bone balance adjustment algorithm 760 may predict a change in laxity of the soft tissue envelope, and adjust (e.g., reduce) the bone resection parameters 762, implant parameters 764, and/or other parameters of the procedure plan 620.

The soft tissue laxity of a joint resulting from removal of an osteophyte may be created in both flexion and extension of the joint. Instead of the soft tissue being “tented” by an osteophyte by, for example, having to travel around the osteophyte, the soft tissue envelope may be lengthened by osteophyte removal in a flexed and/or extended position. When one or more posterior osteophytes are removed, this removal may create laxity in a soft tissue envelope and/or ligament that extended around and/or over the removed osteophyte. In some cases, a posterior osteophyte my effect extension and flexion of one or more ligaments. In other cases, a posterior osteophyte may only effect extension of the one or more ligaments. In some examples, a posterior osteophyte may effect a fraction of a flexion gap and a fraction of an extension gap.

In some examples, the length of the ligament may be measured as it wraps around the osteophyte, and then the joint may be modeled without the osteophyte to predict the original ligament path and/or laxity. An amount of laxity created may correlate to a dimension of the osteophyte over which the soft tissue extends, such as a length, a depth, height, or a combination thereof of the osteophyte. In some other examples, the cross-section of the osteophyte may be calculated to determine the amount of laxity once the osteophyte is removed from the model. In another example, displacement length and/or displacement volume of the ligament required to traverse the osteophyte, before removal in a generated model, may be measured. This dimension (e.g., depth) may be approximated by a surface area (e.g., in millimeters squared or mm²) shown in a predetermined (e.g., sagittal) view of an image, such as a CT image. In some examples, the path of the ligament may be measured around the osteophyte. Once the osteophyte is removed in the model, a path distance of the ligament before and after removal may be determined by comparing the depth, height, and path distance of the ligament to calculate a laxity of the soft tissue. In some examples, a change in laxity may be determined using the attachment points and the path of the one or more ligaments in a model without osteophytes. Attachment points of the ligament may be, for example, entheses such as insertion sites, osteotendinous junctions, osteoligamentous junctions, musculotendinous junctions, and/or other junctions of the ligament and another anatomical portion of the patient.

In some examples, to develop the procedure plan 620 based on the bone balancing system 600, the bone balancing system 600 may compare a predicted path of a ligament with one or more osteophytes present on the target bone (described above), with a second path of a ligament based on the removal of the one or more osteophytes to determine a change in the laxity of a ligament. To determine the change in laxity based on comparing the ligament path with the osteophyte to a proposed ligament path after the osteophyte has been removed, the cross-sectional area of the osteophyte may be used (described further below).

The bone balance adjustment algorithm 760 may be configured to anticipate the laxity created by osteophyte removal, and may determine an alteration of bone resection parameters 762 and/or implant parameters 764 (e.g., thickness of a tibial liner, recommendation for an implant, etc.) to accommodate the increased laxity. The bone balance adjustment algorithm 760 may analyze a predetermined (e.g., sagittal) view of a joint (e.g., knee joint), identify, based on imaging data and/or acquired images of a sagittal view (or other view) using the image acquisition device 610, one or more osteophytes that may not be readily removed before making bone cuts (e.g., posterior osteophyte), determine a surface area of each identified osteophyte, and determine an amount of laxity based on the determined surface area.

The determination may be based on a learned relationship and/or a linear equation. As an example of the linear equation, the bone balance adjustment algorithm 760 may execute a simple y=mx+b formatted equation, where “x” is the surface area in mm²; “y” is the change in a bone resection depth in millimeters or mm, a change in bone resection angle or slope in degrees, or is the change in an implant thickness in mm; m is a multiplier (e.g., input by the practitioner, learned, and/or refined based on results); and b is a constant. For example, in the context of a knee joint, “b” may be 0 and/or adjusted based on preoperative data 720 (e.g., patient data 724, procedure plan 620), and “m” may be in a range of 0.004-0.006 (e.g., 0.005, 0.0055, or 0.0056), but aspects disclosed herein are not limited. For example, “m” and “b” may be different values for different joints and/or adjusted based on patient data. “M” and “b” may also be further adjusted based on learned relationships (e.g., gender) or other preoperative data 720 (e.g., cartilage condition or disease progression), predictions (e.g., cartilage loss or predicted disease progression), intraoperative data, etc. A sign of “m” may depend on an osteophyte symmetry and/or a type of parameter being adjusted. For example, where “y” indicates a bone resection depth, “m” may be negative to indicate a decrease in bone resection with respect to that of the initial procedure plan 620. Where “y” indicates an implant thickness, “m” may be positive to indicate an increase in implant thickness with respect to that of the initial procedure plan 620. Where “y” indicates a bone resection angle or slope, the sign of “m” may depend on whether an osteophyte at one side of the joint is greater than or less than an osteophyte at an opposite of the joint, as will be described in more detail hereinafter.

For example, the bone balance adjustment algorithm 760 may determine that the removal of an osteophyte having a relevant surface area of 90 mm² will result in 0.5 mm of soft tissue laxity based on a linear relationship of 0.5 mm per every 90 mm² and/or 0.1 mm per every 18 mm² (e.g., 180 mm² will result in 1.0 mm of soft tissue laxity, 270 mm² will result in 1.5 mm of soft tissue laxity, 18 mm² will result in 0.1 mm of soft tissue laxity, etc.). In some examples and/or for certain increments, the linear relationship may be simplified and/or rounded up to 0.5 mm per every 100 mm² and/or 0.1 mm per every 20 mm². In other examples, a nonlinear relationship may be used, such as an exponential correlation between osteophyte surface area and soft tissue laxity.

In some other examples, a non-linear equation may be used for determination of the soft tissue laxity using one or more parameters of the detected osteophyte. A non-linear regression may relate two variables in a non-linear (e.g., curved) relationship. The non-linear regression may be created through a series of approximations. The series of approximations may be determined using a Gauss-Newton method, Levenberg-Marquardt method, the like, or a combination thereof. Machine learning may be used to generate a plurality of models to assist with the series of approximations to generate the non-linear regression data, to determine an estimated soft tissue laxity or other parameter associated with a processed medical image.

The bone balance adjustment algorithm 760 may be configured to determine an equation and/or refine the equation based on outcomes 650. For example, while using a linear equation, the bone balance adjustment algorithm 760 may multiply 0.0055 or 0.0056 by the surface area to determine a predicted laxity and/or change in bone resection parameter 762 and/or change in implant parameter 764, but aspects disclosed herein are not limited to this multiplier, which may be adjusted and refined. In some other examples, the bone balance adjustment algorithm 760 may be configured to determine a non-linear equation and/or refine the non-linear equation based on outcomes of 650. The bone balance adjustment algorithm 760 may also store an index of surface areas and adjustments (e.g., in a table or database), which may be updated and/or refined based on the outcomes 650.

Certain dimensions of the osteophyte, such as width, may not be important as the depth for soft tissue laxity because the soft tissue laxity may correlate with a length gained by the soft tissue not having to travel around the osteophyte. For convenience of description, a distance traveled by a hiker hiking over a mountain may primarily depend on two dimensions: (1) a height of the mountain, and (2) a dimension in a forward direction traveled by the hiker. However, a left-right width of the mountain may not be relevant. Similarly, here, only two dimensions may be relevant to the “distance” covered by the soft tissue, while a width may not be relevant. For example, the ligament 204 in FIGS. 2 and 3 may be stretched primarily in the X and Z directions, but not in a direction into and out of the page. The bone balance adjustment algorithm 760 may therefore not need to approximate all dimensions or a volume of identified osteophytes, and may make a determination primarily based on two dimensions or a determined area. The predetermined view may therefore be a two-dimensional image facing the relevant dimensions to depict an extension of the soft tissue and osteophyte in the relevant dimensions. In some other examples, the predetermined view may be a three-dimensional model which may be manipulated by a user. In the context of a posterior osteophyte in a knee joint, this predetermined view may be the sagittal view.

The bone balance adjustment algorithm 760 may also consider a position of the osteophyte planned to be removed to predict the resulting laxity and/or direction of laxity in the soft tissue and/or other bone resection parameters 762. For example, if an osteophyte is removed from a posterolateral femur or a posterolateral tibia, then a soft tissue laxity may be lateral. If the osteophyte is removed from a posteromedial femur or a posteromedial tibia, then the soft tissue laxity may be medial. If there are symmetric posterolateral and posteromedial femoral or tibial osteophytes, then the soft tissue laxity may be global. If there are asymmetric posterolateral and posteromedial femoral or tibial osteophytes, then the soft tissue laxity may be asymmetric.

The bone resection parameters 762 may include angular adjustments and/or slopes determined based on the determined surface area of the osteophyte and/or the determined laxity. For example, if the bone balance adjustment algorithm 760 determines that an osteophyte of a joint is medial, the bone balance adjustment algorithm 760 may determine that removal of the medial osteophyte will create laxity of a medial gap of the joint in flexion and extension. Because some may disagree with the extent of laxity created in flexion in certain joints, in some examples, the bone balance adjustment algorithm 760 may be adjusted and/or consider a practitioner's preference and may determine that removal of the medial osteophyte will create laxity of the medial gap of the joint primarily in extension, but create less laxity (or none at all) in flexion. For example, the bone balance adjustment algorithm 760 may change a type of bone to which less resection is recommended (e.g., tibia to femur).

In the context of a knee joint, the bone balance adjustment algorithm 760 may determine an alteration of a tibial bone cut in the procedure plan 620 in anticipation of the determined laxity of the medial gap based on the determined surface area of the medial osteophyte. An angular adjustment may correspond to the predicted laxity. In some examples, the angular adjustment to the procedure plan 620 may include modifying the bone resection and/or the resection angle values associated with planned surgical cuts or other surgical parameters. By changing the resection plane and/or angle values, the joint may be adjusted to compensate for the removal of an osteophyte while maintaining target laxity. In some examples, the procedure plan 620 may include modifying resection depth and/or resection angle, modifying joint positioning, or both to adjust the implant-to-bone and/or implant-to-implant arrangement. For example, if the bone balance adjustment algorithm 760 determines that the surface area of the medial osteophyte is 90 mm², the bone balancing system may determine a bone resection parameter 762 of 0.5° of tibial valgus (or 0.5° of tibial valgus per 0.5 mm of predicted laxity, or 0.1° of tibial valgus per 0.1 mm of predicted laxity) based on a linear relationship of 0.5° per every 90 mm² and/or 0.1° per every 20 mm² (e.g., 180 mm² will result in 1.0° of tibial valgus, 270 mm² will result in 1.5° of tibial valgus, 20 mm² will result in 0.1° of tibial valgus, etc.). For example, the bone balancing system 600 may multiply 0.0055 or 0.0056 by the surface area to determine a predicted angle or slope (e.g., tibial valgus), but aspects disclosed herein are not limited to this multiplier, which may be adjusted and refined. The bone balance adjustment algorithm 760 may also store an index of surface areas and adjustments, which may be updated and/or refined based on the outcomes 650.

In some embodiments, the bone balance adjustment algorithm 760 may calculate the amount of bone that needs to be resected, and appropriate angle of resection, to adjust for the laxity caused by the removal of the medial osteophyte or bony protrusions such as a posteromedial bony flare. In some examples, a reduction osteotomy is performed by removing a bony protrusion by using a tibial trial as a reference. In some embodiments, the bone balance adjustment algorithm 760 may calculate the amount of bone that needs to be resected after removing both a posteromedial bony flare and a medial osteophyte, to extend the relaxation of the medial soft tissue sleeve beyond what can be achieved by removing medial osteophytes alone. In some examples, correction of varus deformity with medial reduction osteotomy results in a 1° correction for every 2 mm of bone removed. By identifying the correlation between removal of bone and correction of ligament deformity, the risk of over release of soft tissue may be reduced.

Similarly, if the bone balance adjustment algorithm 760 determines that an osteophyte of a joint is lateral, the bone balance adjustment algorithm 760 may determine that removal of the lateral osteophyte will create laxity of a lateral gap of the joint in flexion and extension. Because some may disagree with the extent of laxity created in flexion, in some examples, the bone balance adjustment algorithm 760 may be adjusted and/or consider a practitioner's preference and may determine that removal of the medial osteophyte will create laxity of the medial gap of the joint primarily in extension, but create less laxity (or none at all) in flexion. In some examples, if a surgeon believes that the laxity created by removing the osteophyte will have similar effect on the extension and flexion gaps, then anticipated laxity may be corrected by removing less bone from the tibia. For example, removal of a posterior medial osteophyte will create medial laxity in flexion and extension, therefore the surgeon resects less medial tibial bone. Similarly, removal of a posterior lateral osteophyte will create lateral laxity in flexion and extension, therefore the surgeon resects less lateral tibial bone. If symmetric medial and lateral osteophytes are removed, global laxity will result requiring the surgeon to proximalize the tibia. For surgeons who believe that posterior osteophyte removal results in laxity in extension only, then the strategy is to resect less distal femur on the affected side, or to distalize the femoral cut in the setting of symmetric posterior osteophytes, or to asymmetrically distalize the distal femur in the setting of asymmetric medial and lateral posterior femoral osteophytes.

In the context of a knee joint, the bone balance adjustment algorithm 760 may determine an alteration of a tibial bone cut in the procedure plan 620 in anticipation of the determined laxity of the lateral gap based on the determined surface area of the lateral osteophyte. An angular adjustment may correspond to the predicted laxity. For example, if the bone balance adjustment algorithm 760 determines that the surface area of the lateral osteophyte is 90 mm², the bone balancing system may determine a bone resection parameter 762 of 0.5° of tibial varus (or 0.5° of tibial varus per 0.5 mm of predicted laxity) based on a linear relationship of 0.5° per every 90 mm² and/or 0.1° per every 20 mm² (e.g., 180 mm² will result in 1.0° of tibial varus, 270 mm² will result in 1.5° of tibial varus, 20 mm² will result in 0.1° of tibial varus). For example, the bone balance adjustment algorithm 760 may multiply 0.0055 or 0.0056 by the surface area to determine a predicted angle or slope (e.g., tibial varus), but aspects disclosed herein are not limited to this multiplier, which may be adjusted and refined. The bone balance adjustment algorithm 760 may also store an index of surface areas and adjustments, which may be updated and/or refined based on the outcomes 650.

If the bone balance adjustment algorithm 760 identifies symmetric osteophytes medially and laterally of a joint, then the bone balance adjustment algorithm 760 may determine that removal of these symmetric osteophytes will create global laxity in extension and/or flexion of the joint. In the context of a knee joint, the bone balance adjustment algorithm 760 may determine an alteration of a tibial bone in anticipation of the determined laxity based on the determined surface area of the medial osteophyte. For example, if the bone balance adjustment algorithm 760 determines that the surface area of each of the medial and lateral osteophytes are 90 mm², the bone balance adjustment algorithm 760 may determine a tibial resection of the procedure plan 620 to be 0.5 mm less tibial resection based on a linear relationship of 0.5 mm less tibial resection per every 90 mm² and/or 0.1 mm less tibial resection per every 20 mm² (e.g., 180 mm² will result in 1.0 mm less tibial resection, 270 mm² will result in 1.5 mm less of tibial resection, 20 mm² will result in 0.1 mm less of tibial resection, etc.). Alternatively, bone balance adjustment algorithm 760 may determine a liner used in the procedure plan 620 should have a thickness of 0.5 mm or more (note: linear thickness available in 1 mm increments) and/or an increased thickness based on the linear relationship as an implant parameter 764.

If the bone balancing system 600 identifies medial and lateral osteophytes of different size, then the bone balance adjustment algorithm 760 may determine a greater laxity and/or adjustment to the procedure plan 620 (e.g., tibial bone cut) on a side having the larger osteophyte. For example, the bone balancing system 600 may determine a difference in a surface area of a medial osteophyte and a surface area of a lateral osteophyte. Where the medial osteophyte is larger than the lateral osteophyte and the difference in surface area is 90 mm², the bone balance adjustment algorithm 760 may determine that the procedure plan 620 should include a bone resection parameter 762 of 0.5° of tibial valgus based on a linear relationship of 0.5° per every 90 mm² and/or 0.1° per every 20 mm² (e.g., 180 mm² will result in 1.0° of tibial valgus, 270 mm² will result in 1.5° of tibial valgus, 20 mm² will result in 0.1° of tibial valgus, etc.). Similarly, where the lateral osteophyte is larger than the medial osteophyte and the difference in surface area is 90 mm², the bone balance adjustment algorithm 760 may determine that the procedure plan 620 should include a bone resection parameter 762 of 0.5° of tibial varus based on a linear relationship of 0.5° per every 90 mm² and/or 0.1° per every 20 mm² (e.g., 180 mm² will result in 1.0° of tibial varus, 270 mm² will result in 1.5° of tibial varus, 20 mm² will result in 0.1° of tibial valgus etc.). etc.

FIG. 8 illustrates an exemplary graphical user interface of the bone balancing system 600 displayed on the display 630. Referring to FIGS. 6-8, a practitioner (e.g., surgeon) may use a computer or other system of the bone balancing system 600 to analyze one or more images of a patient's anatomy 802 (e.g., knee joint) and/or an initially determined procedure plan 620. The one or more images of the patient's anatomy 802 and/or the procedure plan 620 may be included in a case file, patient folder, etc. that is selectable by the practitioner. The one or more images of the patient's anatomy 802 may have been acquired using an image acquisition device 610 and/or with the patient in various poses. The bone balancing system 600 may analyze the one or more images of the patient's anatomy 802 to detect and/or scan for osteophytes. If one or more osteophytes are detected, the bone balancing system 600 may provide a notification or alert 804 (e.g., pop-up or dialogue box) that osteophytes were detected. In some examples, the bone balancing system 600 may determine that the osteophytes are planned and/or should be planned to be removed in the procedure plan 620, but that removal will be made after bone cuts and/or will affect soft tissue laxity. In such an example, the notification 804 may include text 806 (e.g., “Make adjustments to procedure plan?”) offering to make adjustments to the procedure plan 620 based on the detected osteophytes and present one or more selectable user inputs 808 (e.g., “yes” or “no” buttons) that the practitioner may select. If the practitioner commands and/or indicates, using the selectable user inputs 808, adjustments to be made, the bone balancing system 600 may execute the bone balance adjustment algorithm 760 to determine the adjustments to the procedure plan 620 and/or to determine a new procedure plan 620.

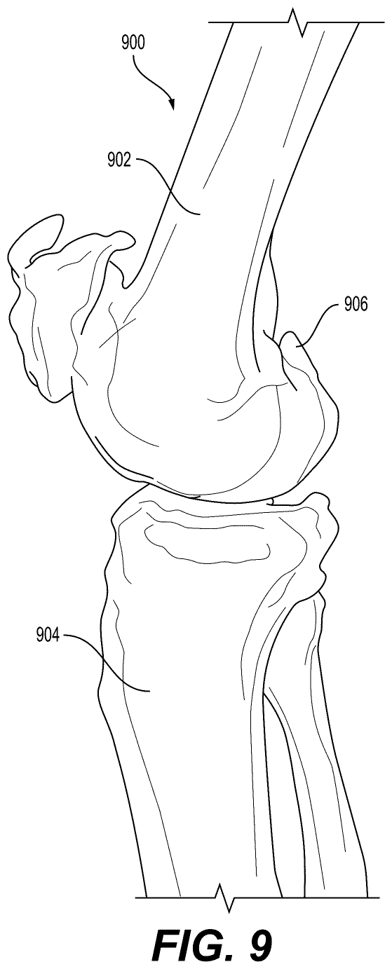

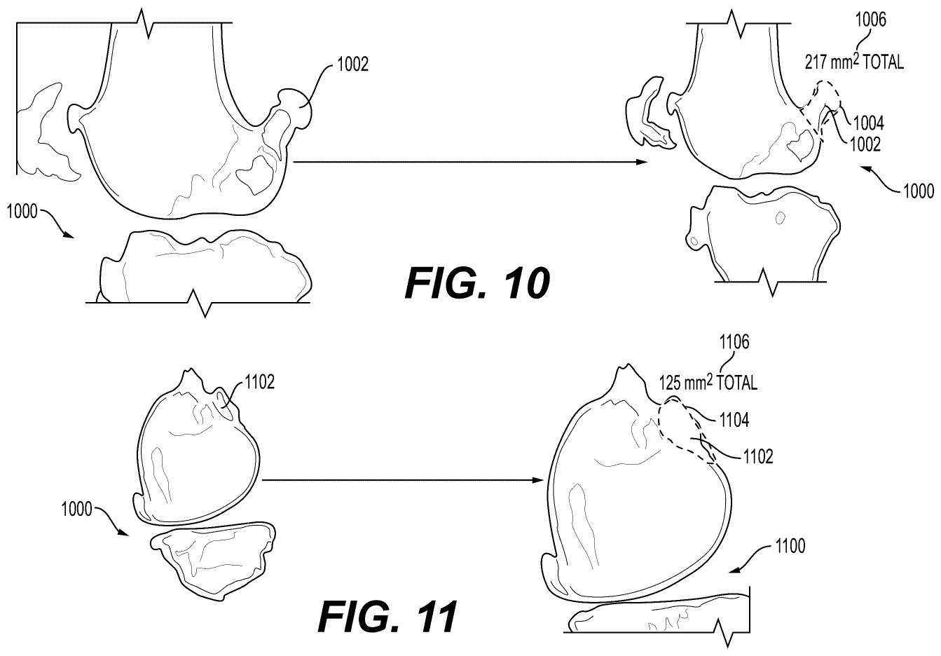

FIG. 9 illustrates an image 900 of a patient's anatomy including a femur 902, a tibia 904, and an osteophyte 906. Referring to FIGS. 6-9, a practitioner may select (e.g., using a computer having the display 630) the image 900 among a plurality of images to display. The bone balancing system 600 may detect the osteophyte 906 and determine that the osteophyte 906 will likely affect soft tissue laxity after removal. The bone balancing system 600 may determine that the osteophyte 906 is inaccessible until one or more bone cuts is made to the femur 902 and/or tibia 904. FIGS. 10-11 show two examples of detection and calculation of surface area by the bone balancing system 600. FIG. 10 may show an example based on the osteophyte 106 shown in FIG. 1, and FIG. 11 may show an example based on the osteophyte 906 shown in FIG. 9.

Referring to FIGS. 6-7 and 10, the bone balancing system 600 may identify an image 1000 that displays a largest extent of a detected osteophyte 1002 in a predetermined dimension. In FIG. 10, the image 1000 may show a sagittal view of a knee joint and display a largest protrusion of the osteophyte 1002. The bone balancing system 600 may analyze the image 1000 and display a border or outline 1004 of the osteophyte 1002. For example, the bone balancing system 600 may execute a segmenting or thresholding technique, but aspects disclosed herein are not limited. Alternatively or in addition thereto, a practitioner may select an area of the osteophyte 1002 and/or draw the border or outline 1004 (e.g., using a mouse) for the bone balancing system 600 to analyze. The bone balancing system 600 may calculate and/or display a surface area 1006 of the osteophyte 1002, which may be based on the border or outline 1004. The surface area 1006 may be used to determine or predict a soft tissue laxity of a soft tissue (e.g., ligament) that extends over the osteophyte 1002. Similarly, referring to FIGS. 6-7 and 11, the bone balancing system 600 may identify an image 1100 that displays a largest extent of a detected osteophyte 1102 in a predetermined dimension. In FIG. 11, the image 1100 may show a sagittal view of a knee joint and display a largest protrusion of the osteophyte 1102. The bone balancing system 600 may analyze the image 1100 and display a border or outline 1104 of the osteophyte 1102. For example, the bone balancing system 600 may execute a segmenting or thresholding technique, but aspects disclosed herein are not limited. Alternatively or in addition thereto, a practitioner may select an area of the osteophyte 1102 and/or draw the border or outline 1104 (e.g., using a mouse) for the bone balancing system 600 to analyze. The bone balancing system 600 may calculate and/or display a surface area 1106 of the osteophyte 1102, which may be based on the border or outline 1104. The surface area 1106 may be used to determine or predict a soft tissue laxity of a soft tissue (e.g., ligament) that extends over the osteophyte 1102.

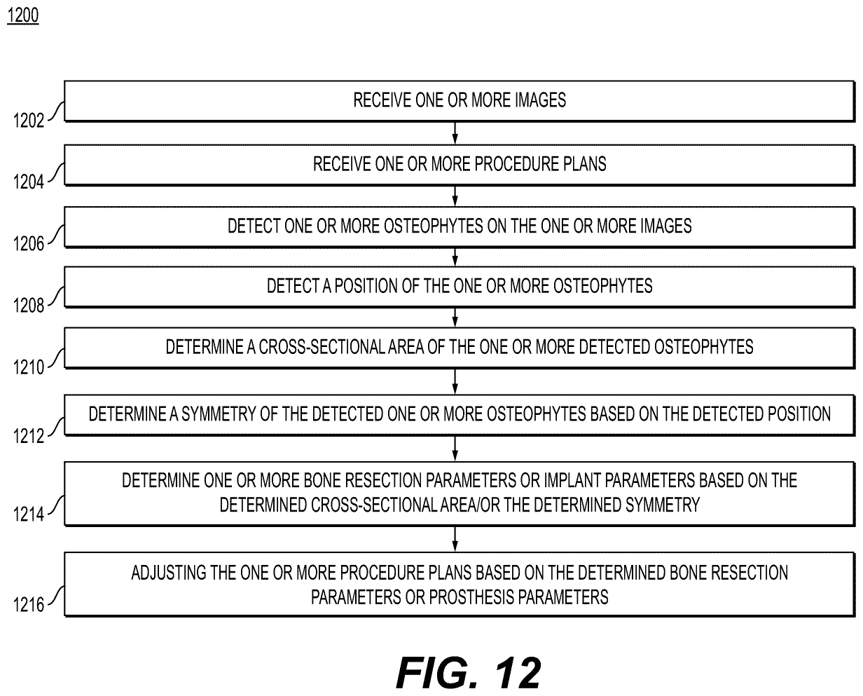

FIG. 12 illustrates an exemplary preoperative method 1200 for adjusting a preoperative or procedure plan based on detected osteophytes. Referring to FIG. 12, the method 1200 may be implemented by the bone balancing system 600 (e.g., using bone balance adjustment algorithm 760) and/or the practitioner. The method 1200 may include a step 1202 of receiving one or more images of a patient's anatomy, such as one more CT scans, ultrasound images, or X-rays. Step 1202 may include receiving the one or more images into electronic storage from, for example, an image acquisition device or other system. The method 1200 may include a step 1204 of receiving one or more procedure plans including one or more bone cuts. The procedure plan may also include an implant configured to be received in a bone at the one or more bone cuts. Step 1204 may include receiving the one or more procedure plans from a practitioner via manual entry, from a remote system that determined the one or more procedure plans, from a memory, and/or may include determining the one or more procedure plans using, for example, the preoperative data 720 described with reference to FIG. 7.

The method 1200 may include a step 1206 of detecting one or more osteophytes on the one or more received images. Step 1206 may include using image processing techniques to identify osteophytes and/or machine learning systems trained to detect osteophytes (e.g., autonomous segmentation and/or calculation of osteophyte size and location). Alternatively or in addition thereto, step 1206 may include receiving input from a practitioner (e.g., via a computer) and/or an annotation indicating one or more osteophytes on the one or more received images. Step 1206 may include detecting osteophytes and identifying whether each detected osteophyte is accessible and/or inaccessible for removal before bone cuts. Step 1206 may include determining whether each detected osteophyte would be inaccessible even after bone cuts. If, in step 1206, it is determined that a detected osteophyte would be inaccessible even after bone cuts (or highly difficult to access), then the procedure plan and/or predicted soft tissue may be adjusted to account for not removing such osteophyte (such that there may be less of a change to soft tissue laxity).

The method 1200 may include a step 1208 of detecting a position or location of the one or more detected osteophytes. Step 1208 may include detecting a side or compartment (e.g., medial or lateral) of the one or more detected osteophytes relative to a bone, and/or may include determining a coordinate or other positional parameter of the one or more detected osteophytes relative to the bone and/or each other where more than one osteophyte is detected. Step 1208 may include detecting the position using image recognition and/or processing techniques and/or analyzing a plurality of images of the same joint. Alternatively or in addition thereto, step 1208 may include receiving input from a practitioner (e.g., via a computer) and/or an annotation indicating the position of the one or more osteophytes on the one or more received images.

The method 1200 may include a step 1210 of determining a cross-sectional or surface area of the one or more detected osteophytes. Step 1210 may include determining the surface area using image recognition and/or processing techniques and/or analyzing a plurality of images of the same joint. For example, step 1210 may include determining or detecting a boundary of the bone (e.g. tibia, femur), the osteophyte, or both on an image, and calculating an area within the boundary. When the boundary of the bone, osteophyte, or both within an image are identified, the image data may be used to create a model of the joint. In some examples, the joint model may include one or more osteophytes, or may be created without the one or more osteophytes. In one example, the boundary and/or volume of each of the one or more osteophytes may be subtracted from the image data to create an osteophyte-free model. Alternatively or in addition thereto, step 1210 may include receiving input from a practitioner (e.g., via a computer) and/or an annotation indicating the surface area of the one or more osteophytes on the one or more received images. The determined cross-sectional area may be output on a display or other output system.

In some examples, step 1210 may include identifying an image among a plurality of received images that best reflects the surface area of the osteophyte. For example, step 1210 may include identifying one or more images, among a plurality of received images, that show the patient's anatomy (e.g., joint) across a first predetermined dimension and a second predetermined dimension over which the soft tissue may extend. In the context of a knee joint, the one or more identified images may be sagittal views of the knee joint. Step 1210 may further include identifying an image, among the identified images reflecting the first and second predetermined dimensions (e.g., sagittal view), that shows the greatest extent of the osteophyte in one of the first and second predetermined dimensions. In some examples, step 1210 may include determining a surface area of the detected osteophyte based on a plurality of images of a same view (e.g., sagittal view) that include the osteophyte, and identifying a maximum surface area calculation, a mean surface area calculation, and/or a median surface area calculation. In some examples, step 1210 may include determining a surface area of the detected osteophyte based on a plurality of images and/or determining a volume of the detected osteophyte, but the algorithm may be simplified by considering surface area in the two most important dimensions, and disregarding a dimension of the osteophyte in a dimension that does not affect soft tissue laxity. In other examples, step 1210 may include determining a height or depth of the osteophyte in one of the predetermined dimensions in place of or in addition to the surface area of the detected osteophyte in two dimensions where, for example, the surface area cannot be calculated (e.g., due to poor image quality). In such a situation, the bone balance adjustment algorithm 760 may be adjusted to account for single dimension to approximate the effect on soft tissue laxity.

The method 1200 may include a step 1212 of determining a symmetry of the detected osteophytes based on the detected positions and/or determined cross-sectional areas. For example, if, in step 1202, more than one osteophyte is detected, step 1212 may include determining that the osteophytes are symmetric based on their detected positions and sizes and/or asymmetric based on their detected positions and sizes. Step 1212 may include determining that two osteophytes are symmetric if they are of a same size or similar size (e.g., a difference in the determined cross-sectional areas is below a predetermined threshold) and are positioned on opposite sides of a joint. Step 1212 may include determining that two osteophytes are asymmetric if they are of a different size (e.g., a difference in the determined cross-sectional areas is above a predetermined threshold) and/or they occur on a same side of the joint (and/or they occur on sides of the joint that are not opposite). Asymmetry at step 1212 may also be determined based on the detected osteophytes being different distances or positions away from a predetermined axis.

Step 1212 may include using image recognition and/or processing techniques and/or analyzing a plurality of images of the same joint. Alternatively or in addition thereto, step 1212 may include receiving input from a practitioner (e.g., via a computer) and/or an annotation indicating the symmetry of osteophytes on the one or more received images.

The method 1200 may include a step 1214 of determining one or more bone resection parameters or implant parameters based on the determined cross-sectional area/or the determined symmetry. Step 1214 of determining the one or more bone resection parameters and/or implant parameters may include determining a change in a bone resection parameter and/or implant parameter included in the initially received procedure plan, such as adjusting the angle of resection and/or adjusting the resection angle values to adjust the joint. Step 1214 may also include determining a change in and/or amount of a soft tissue laxity after removal of the detected osteophytes. The determinations made at step 1214 may be output on a display or other output system, for example, as a text of instructions and/or list of steps, as a chart, visually on a virtual bone model, etc.

Step 1214 may include executing the bone balance adjustment algorithm 760, which may implement one or more linear relationships, as previously described with respect to FIG. 6. The linear relationship may be input and/or refined by the practitioner. Alternatively or in addition thereto, the linear relationship may be learned or refined through assessing multiple patients. In some examples, the linear relationship may be stored and/or implemented using an index database using piecemeal relationships of known surface area amounts, known symmetry, and corresponding changes to the one or more bone resection parameters or implant parameters. In some examples, the linear relationship may include an equation having a multiplier or slope that is multiplied by the surface area to result in the one or more bone resection parameters or implant parameters.

In some other examples, the bone balance adjustment algorithm 760 may implement one or more non-linear relationships. The non-linear relationship may be input and/or refined by the practitioner. Alternatively or in addition thereto, the non-linear relationship may be learned or refined through assessing multiple patients. In some examples, the non-linear relationship may be stored and/or implemented using an index database using piecemeal relationships of known surface area amounts, known symmetry, and corresponding changes to the one or more bone resection parameters or implant parameters. In some examples, the non-linear relationship may include an equation having a multiplier or slope that is multiplied by the surface area (e.g. of one or more osteophytes, ligaments, bones, etc.) in target area(s) to result in the one or more bone resection parameters or implant parameters.

The method 1200 may include a step 1216 of adjusting the one or more procedure plans based on the determined bone resection parameters and/or implant parameters from step 1214. Step 1216 may be performed by the bone balance adjustment algorithm 760. Alternatively or in addition thereto, the practitioner may manually adjust the procedure plan based on the determined bone resection parameters and/or implant parameters from step 1214.

As an example, in steps 1214 and 1216, the bone balance adjustment algorithm 760 may determine that the removal of an osteophyte having a determined surface area (e.g., 90 mm² or 20 mm²) will result in determined amount of soft tissue laxity (e.g., 0.5 mm or 0.1 mm), that a bone resection parameter should decrease by a predetermined amount (e.g., 0.5 mm or 0.1 mm) or that an implant parameter (e.g., thickness) should increase by a predetermined amount (e.g., 0.5 mm or 0.1 mm), and/or, based on a symmetry, that a bone resection angle should change in a predetermined direction or orientation (e.g., varus or valgus) by a predetermined amount (e.g., 0.5° or) 0.1°. Examples of these adjustments are described in more detail with respect to FIG. 6 and FIGS. 13-14. After steps 1214 and 1216, the procedure plan may be updated with the adjustments so that the surgeon may perform the procedure plan during surgery. The entire method 1200 may be performed before the procedure and before any bone cuts are made. Alternatively or in addition thereto, steps 1214 and/or 1216 may be repeated based on new intraoperative information received regarding osteophytes (e.g., those identified during surgery but not picked up by the received images), their positions, and/or their size. The determinations made during method 1200 may be output to a display, for example as a virtual model, a list of steps, as a finalized procedure plan, etc. In some examples, the determinations and/or finalized procedure plan may be output as a set of programmed instructions configured for execution by a robotic platform 640 (e.g., CNC instructions) that may assist with bone cuts or other steps in a medical procedure.

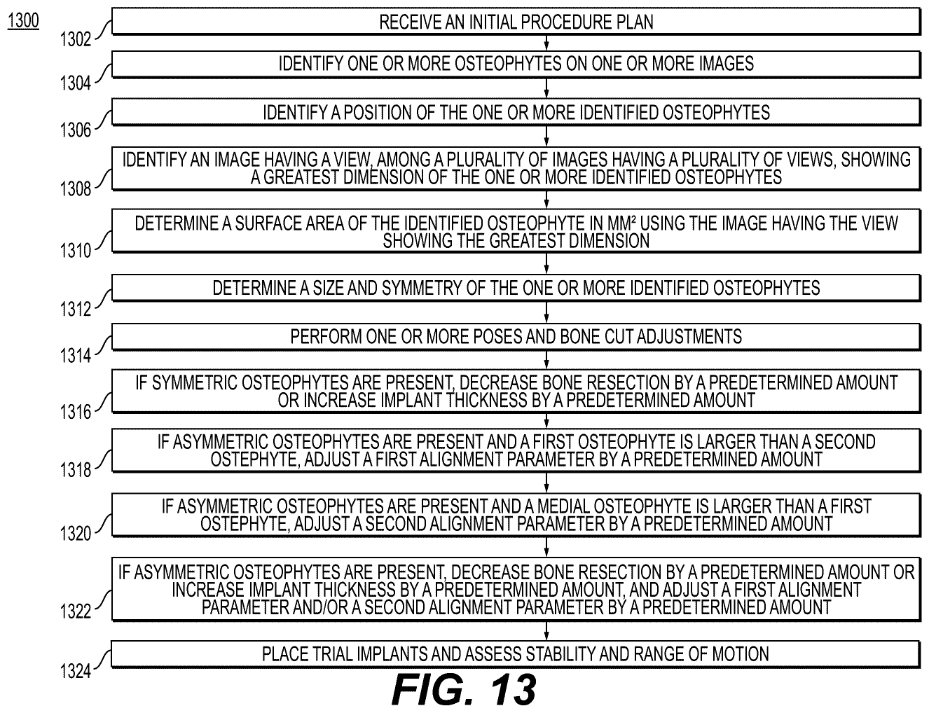

FIG. 13 illustrates an exemplary preoperative and/or intraoperative method 1300 for adjusting a procedure plan based on detected osteophytes. Some of the steps of method 1300 are similar to steps of method 1200, and for convenience of description, repetitive or similar portions of the description of certain steps of method 1300 may be omitted.

The method 1300 may include a step 1302 of receiving an initial procedure plan, a step 1304 of identifying or detecting one or more osteophytes on one or more received images of a joint, and a step 1306 of identifying a position of the one or more identified osteophytes. In addition the method 1300 may include a step 1308 identifying an image having a view, among a plurality of images having a plurality of views, showing a greatest dimension of the one or more identified osteophytes (e.g., sagittal view). Steps 1302 through 1308 may include using image recognition and/or processing techniques and/or analyzing a plurality of images of the same joint. Alternatively or in addition thereto, steps 1302 through 1308 may include receiving input from a practitioner (e.g., via a computer) and/or an annotations (e.g. on the one or more received images).

The method 1300 may include a step 1310 of determining a surface and/or cross-sectional area of the one or more identified osteophytes in mm² using the image identified in step 1308 having the view showing the greatest dimension. Step 1310 may include may include determining the surface area and/or cross-sectional area using image recognition and/or processing techniques and/or analyzing a plurality of images of the same joint. Alternatively or in addition thereto, step 1310 may include receiving input from a practitioner (e.g., via a computer) and/or an annotation indicating the surface area, and/or receiving the surface area from input.

The method 1300 may include a step 1312 of determining a size and symmetry of the one or more identified osteophytes. Determining the size at step 1312 may include using the surface area determined in step 1310 and/or determining a size of another parameter (e.g., volume) of the osteophyte using, for example, image processing techniques (e.g., autonomous segmentation and/or calculation of osteophyte size and location). For more than one osteophyte, determining the size at step 1312 may include determining a difference in size among osteophytes. Determining the symmetry at step 1312 may be based on the determined position at step 1306 and a determined difference in size of the osteophytes.

The method 1300 may include a step 1314 of performing one or more poses. Step 1314 may include stressing soft tissues of a joint in both flexion and extension by applying predetermined (e.g., varus and valgus forces). Images and/or other preoperative data 720 may be acquired at each pose. The poses may be performed at step 1314 according to a practitioner preference. Step 1314 may also include determining a size and shape of flexion and extension gaps. Step 1314 may include adjusting the initial procedure plan received at step 1302 based on the determined size and shape of the flexion and extension gaps. Alternatively or in addition thereto, step 1314 may be performed before step 1302, and the received procedure plan at step 1302 may include parameters and/or adjustments determined based on the performed one or more poses. Alternatively or in addition thereto, step 1314 may be performed right before a procedure and/or intraoperatively before bone cuts.

The method 1300 may include making further adjustments, as outlined in steps 1316 through 1322. In step 1316, if, at step 1312, it was determined (e.g., by bone balancing system 600 and/or the practitioner) that symmetric osteophytes are present, the bone balance adjustment algorithm 760 may determine that a bone resection (e.g., tibial and/or femoral resection) in the procedure plan should be decreased by a predetermined amount, or that an implant thickness should be increased by a predetermined amount. In some examples, the adjustment algorithm 760 may determine which types of bone resections (e.g., tibial and/or femoral) should be adjusted and/or may receive a practitioner's input of a type of bone resection. Alternatively or in addition thereto, the adjustment algorithm 760 may receive a practitioner's input related to an expected laxity effect (e.g., an indication that the practitioner expects laxity to be affected in both extension and flexion or expects laxity to be affected primarily in extension) or other practitioner preference, and the adjustment algorithm 760 may determine the type of bone resection based on the practitioner's input.

In step 1318, if, at step 1312, it was determined that asymmetric osteophytes are present and a first (e.g., lateral) osteophyte is larger than a second (e.g., medial osteophyte), the bone balance adjustment algorithm 760 may determine that a first alignment parameter (e.g., bone resection angle in a first direction, or varus) should be adjusted, such as increased, by a predetermined amount. In step 1320, if, at step 1312, it was determined that asymmetric osteophytes are present and the second (e.g., medial) osteophyte is larger than the first (e.g., lateral) osteophyte, the bone balance adjustment algorithm 760 may determine that a second alignment parameter (e.g., bone resection angle in a second direction, or valgus) should be adjusted by a predetermined amount.

In step 1322, if at step 1312, it was determined that asymmetric osteophytes are present, and each osteophyte has a size greater than a predetermined size and/or a difference in sizes of the osteophytes is greater than a predetermined size difference, the bone balance adjustment algorithm 760 may determine a combination of adjustments, such as decreasing a bone resection by a predetermined amount or increase implant thickness by a predetermined amount, and adjust a first alignment parameter and/or a second alignment parameter by a predetermined amount. The determinations made at steps 1312-1322 may be output on a display or other output system, for example, as a text of instructions and/or list of steps, as a chart, visually on a virtual bone model, etc. In some examples, the determinations and/or a finalized procedure plan may be output as a set of programmed instructions configured for execution by a robotic platform 640 (e.g., CNC instructions) that may assist with bone cuts or other steps in a medical procedure.

The method 1300 may include a step 1324 of placing trial implants and assessing stability and range of motion. Step 1324 may be performed intraoperatively after bone cuts are made, and further adjustments may be made intraoperatively.

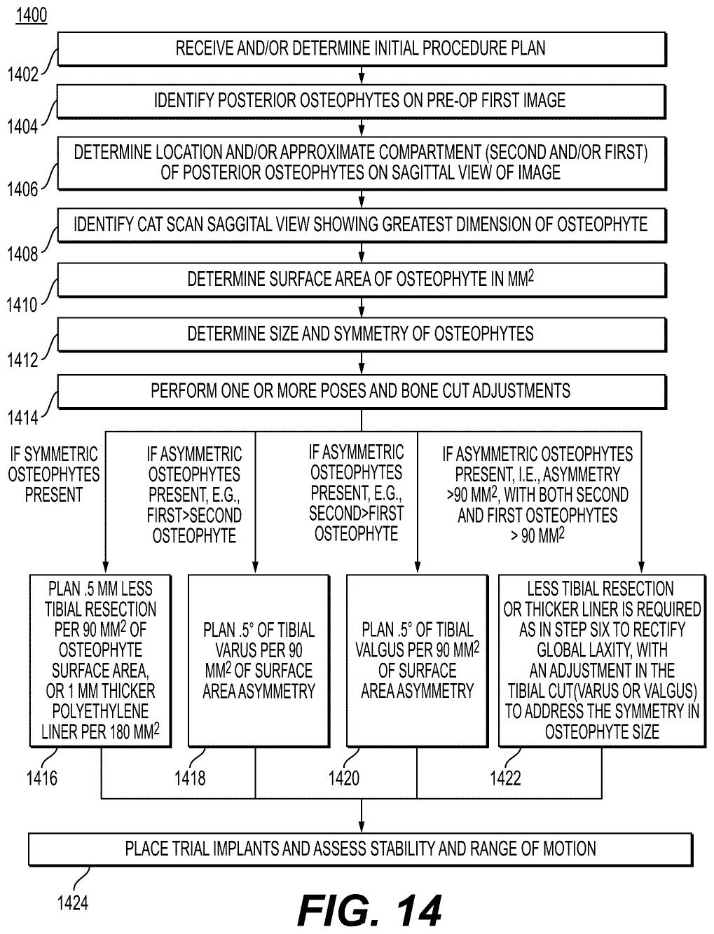

FIG. 14 illustrates an exemplary method 1400 for adjusting a procedure plan for a knee surgery (e.g., total or partial knee arthroplasty) based on detected osteophytes. Some of the steps of method 1400 are similar to steps of methods 1200 and/or 1300, and for convenience of description, repetitive or similar portions of the description of certain steps of method 1400 may be omitted.

The method 1400 may include a step 1402 of receiving an initial procedure plan. The method 1400 may include a step 1404 of identifying or detecting one or more posterior osteophytes on one or more images of a knee joint by, for example, analyzing the one or more images. Step 1404 may include receiving the one or more images. The one or more received images may include one or more preoperative lateral or sagittal view images, which may have been acquired using an X-ray machine, ultrasound, and/or CT scan machine.

The method 1400 may include a step 1406 of identifying a position and/or approximate compartment (e.g., lateral or medial) of the identified posterior osteophytes. Step 1406 may include identifying the position and/or location of the identified posterior osteophytes on one or more sagittal views of an image.

The method 1400 may include a step 1408 of identifying a sagittal image or view among a plurality of images showing a greatest dimension (e.g., depth or height) of the one or more identified posterior osteophytes. Steps 1402 through 1408 may include using image recognition and/or processing techniques and/or analyzing a plurality of images of the same joint. Alternatively or in addition thereto, steps 1402 through 1408 may include receiving input from a practitioner (e.g., via a computer) and/or an annotations (e.g. on the one or more received images).

The method 1400 may include a step 1410 of determining a surface and/or cross-sectional area of the one or more identified posterior osteophytes in mm² using the image identified in step 1408 having the view showing the greatest dimension. The method 1400 may include a step 1412 of determining a size and symmetry of the one or more identified posterior osteophytes. Determining the size at step 1412 may include using the surface area determined in step 1410 and/or determining a size of another parameter (e.g., volume) of the osteophyte using, for example, image processing techniques (e.g., autonomous segmentation and/or calculation of osteophyte size and location). Determining the size at step 1412 may include determining a difference in size of a detected lateral osteophyte and a detected medial osteophyte. Determining the symmetry at step 1412 may be based on the determined location at step 1406 and a determined difference in size of the osteophytes.

The method 1400 may include a step 1414 of performing one or more poses. Step 1414 may include stressing medial and lateral soft tissues of a joint in both flexion and extension by applying varus and valgus forces. Images and/or other preoperative data 720 may be acquired at each pose. Step 1414 may also include determining a size and shape of flexion and extension gaps. Step 1414 may include adjusting the initial procedure plan received at step 1402 based on the determined size and shape of the flexion and extension gaps. Alternatively or in addition thereto, step 1414 may be performed before step 1402, and the received procedure plan at step 1402 may include parameters and/or adjustments determined based on the performed one or more poses. Alternatively or in addition thereto, step 1414 may be performed right before a procedure and/or intraoperatively before bone cuts.

The method 1400 may include making further adjustments, as outlined in steps 1416 through 1422. In step 1416, if, at step 1412, it was determined (e.g., by bone balance system 600 and/or the practitioner) that symmetric osteophytes are present, the bone balance adjustment algorithm 760 may determine that a tibial resection in the procedure plan should be decreased by 0.5 mm per 90 mm² of determined surface area of the determined osteophytes, or that an implant thickness should be increased by 1 mm per a 180 mm² of determined surface area, etc. However, aspects disclosed herein are not limited to these specific linear relationships. For example, based on other preoperative data 720, the bone balance system 600 may determine that the tibial resection should be decreased by 0.5 mm per 95 mm² of determined surface area, or 0.4 mm per 90 mm², etc.

In step 1418, if, at step 1412, it was determined that asymmetric osteophytes are present and a lateral osteophyte is larger than a medial osteophyte, the bone balance adjustment algorithm 760 may determine to adjust a resection angle by 0.5° of tibial varus per 90 mm² of asymmetry in the surface area, or per a 90 mm² of difference between the surface area of the lateral osteophyte and the surface area of the medial osteophyte. A varus tibial cut may be made by fixing a pivot point medially and taking less lateral bone from the tibia.

In step 1420, if, at step 1412, it was determined that asymmetric osteophytes are present and the medial osteophyte is larger than the lateral osteophyte, the bone balance adjustment algorithm 760 may determine to adjust a resection angle by 0.5° of tibial valgus per 90 mm² of asymmetry in the surface area, or per a 90 mm² of difference between the surface area of the lateral osteophyte and the surface area of the medial osteophyte. A valgus tibial cut may be made by fixing a pivot point laterally and taking less medial bone from the tibia.

In step 1422, if at step 1412, it was determined that asymmetric osteophytes are present, that each of the medial and lateral osteophytes have a surface area of greater than 90 mm², and also that a difference in surface area between the medial osteophyte and the lateral osteophyte is greater than 90 mm², then the bone balance adjustment algorithm 760 may determine a combination of adjustments, such as decreasing a resection, increasing implant thickness, and/or adjusting tibial varus and/or valgus. The determinations made at steps 1412-1422 may be output on a display or other output system, for example, as a text of instructions and/or list of steps, as a chart, visually on a virtual bone model, etc. In some examples, the determinations and/or finalized procedure plan may be output as a set of programmed instructions configured for execution by a robotic platform 640 (e.g., CNC instructions) that may assist with bone cuts or other steps in a medical procedure, such as ligament releases (e.g. cutting through or disconnecting a ligament). In some examples, ligament releases may be used with additional bone resection(s). In other examples, only ligament releases may be includes in the procedure plan and not bone resection.

The method 1400 may include a step 1424 of placing trial implants and assessing stability and range of motion. Step 1424 may be performed intraoperatively after bone cuts are made, and further adjustments may be made intraoperatively. Although the method 1400 describes a knee surgery, aspects disclosed herein may be used to make adjustments to other joints (e.g., elbows, hands, spine, ankles, feet, neck, etc.), especially other joints where osteophytes or other growths may form and/or may need to be removed (e.g., to increase mobility and/or decrease pain or impingement).

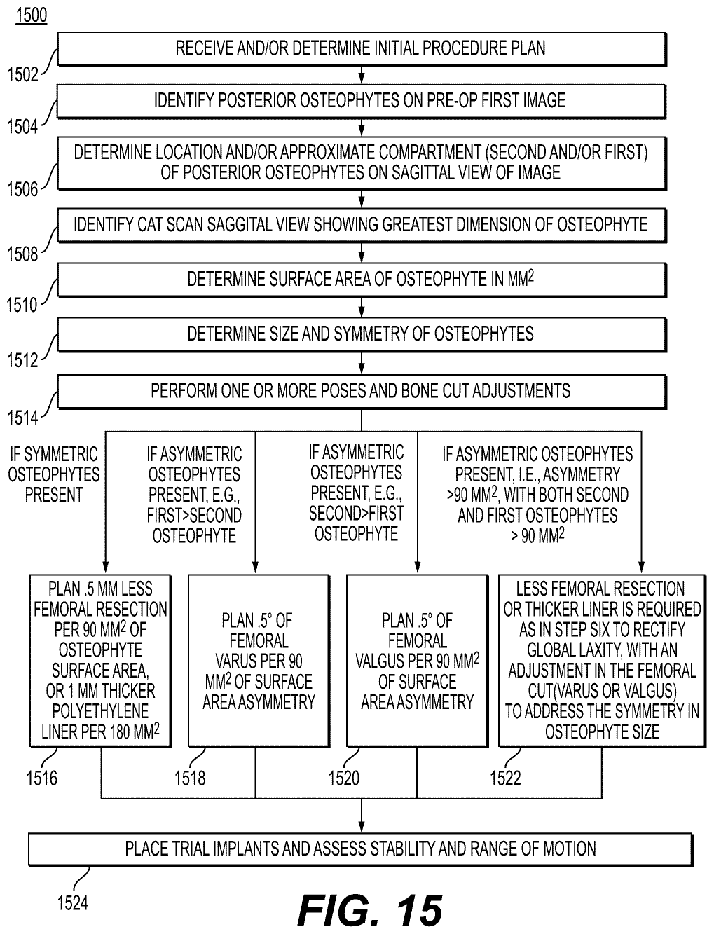

Referring to FIG. 15, some practitioners may disagree with the extent of laxity created in flexion for a particular joint, such as in the context of a knee joint, and the bone balance adjustment algorithm 760 may be adjusted and/or consider a practitioner's preference and may determine that removal of, for example, a medial osteophyte will create laxity of a medial gap of the joint primarily in extension, but create less laxity (or none at all) in flexion. Depending on a practitioner's preference, a method 1500 may be performed for a knee joint instead of or in addition to the previous methods described herein. Some of the steps of the method 1500 are similar to steps of methods 1200, 1300, and/or 1400, and for convenience of description, repetitive or similar portions of the description of certain steps of method 1500 may be omitted. The method 1500 may differ from the method 1400 by predicting adjustments to femoral resection, femoral varus, and/or femoral valgus, rather than tibial resection, tibial varus, and/or tibial valgus.

The method 1500 may include a step 1502 of receiving an initial procedure plan. The method 1500 may include a step 1504 of identifying or detecting one or more posterior osteophytes on one or more images of a knee joint by, for example, analyzing the one or more images. Step 1504 may include receiving the one or more images. The one or more received images may include one or more preoperative lateral or sagittal view images, which may have been acquired using an X-ray machine, an ultrasound, and/or CT scan machine.

The method 1500 may include a step 1506 of identifying a position and/or approximate compartment (e.g., lateral or medial) of the identified posterior osteophytes. Step 1506 may include identifying the position and/or location of the identified posterior osteophytes on one or more sagittal views of an image.

The method 1500 may include a step 1508 of identifying a sagittal image or view among a plurality of images showing a greatest dimension (e.g., depth or height) of the one or more identified posterior osteophytes. Steps 1502 through 1508 may include using image recognition and/or processing techniques and/or analyzing a plurality of images of the same joint. Alternatively or in addition thereto, steps 1502 through 1508 may include receiving input from a practitioner (e.g., via a computer) and/or an annotations (e.g. on the one or more received images).

The method 1500 may include a step 1510 of determining a surface and/or cross-sectional area of the one or more identified posterior osteophytes in mm² using the image identified in step 1508 having the view showing the greatest dimension. The method 1500 may include a step 1512 of determining a size and symmetry of the one or more identified posterior osteophytes. Determining the size at step 1512 may include using the surface area determined in step 1510 and/or determining a size of another parameter (e.g., volume) of the osteophyte using, for example, image processing techniques (e.g., autonomous segmentation and/or calculation of osteophyte size and location). Determining the size at step 1512 may include determining a difference in size of a detected lateral osteophyte and a detected medial osteophyte. Determining the symmetry at step 1512 may be based on the determined location at step 1506 and a determined difference in size of the osteophytes.

The method 1500 may include a step 1514 of performing one or more poses. Step 1515 may include stressing medial and lateral soft tissues of a joint in both flexion and extension by applying varus and valgus forces. Images and/or other preoperative data 720 may be acquired at each pose. Step 1514 may also include determining a size and shape of flexion and/or extension gaps. Step 1514 may include adjusting the initial procedure plan received at step 1502 based on the determined size and shape of the flexion and/or extension gaps. Alternatively or in addition thereto, step 1514 may be performed before step 1502, and the received procedure plan at step 1502 may include parameters and/or adjustments determined based on the performed one or more poses. Alternatively or in addition thereto, step 1514 may be performed right before a procedure and/or intraoperatively before bone cuts.

The method 1500 may include making further adjustments, as outlined in steps 1516 through 1522. In step 1516, if, at step 1512, it was determined (e.g., by bone balance system 600 and/or the practitioner) that symmetric osteophytes are present, the bone balance adjustment algorithm 760 may determine that a femoral resection (e.g., distal femoral resection) in the procedure plan should be decreased by 0.5 mm per 90 mm² of determined surface area of the determined osteophytes, or that an implant thickness should be increased by 0.5 mm per 90 mm² of determined surface area or 1 mm per a 180 mm² of determined surface area, etc. However, aspects disclosed herein are not limited to these specific linear relationships. For example, based on other preoperative data 720, the bone balance system 600 may determine that the femoral resection should be decreased by 0.5 mm per 95 mm² of determined surface area, or 0.4 mm per 90 mm², etc.

In step 1518, if, at step 1512, it was determined that asymmetric osteophytes are present and a lateral osteophyte is larger than a medial osteophyte, the bone balance adjustment algorithm 760 may determine to adjust a resection angle by 5° of femoral varus per 90 mm² of asymmetry in the surface area, or per a 90 mm² of difference between the surface area of the lateral osteophyte and the surface area of the medial osteophyte. A varus femoral cut may be made by fixing a pivot point medially and taking less lateral bone from the distal femur.

In step 1520, if, at step 1512, it was determined that asymmetric osteophytes are present and the medial osteophyte is larger than the lateral osteophyte, the bone balance adjustment algorithm 760 may determine to adjust a resection angle by 5° of femoral valgus per 90 mm² of asymmetry in the surface area, or per a 90 mm² of difference between the surface area of the lateral osteophyte and the surface area of the medial osteophyte. A valgus femoral cut may be made by fixing a pivot point laterally and taking less medial bone from the distal femur.

In step 1522, if at step 1512, it was determined that asymmetric osteophytes are present, that each of the medial and lateral osteophytes have a surface area of greater than 90 mm², and also that a difference in surface area between the medial osteophyte and the lateral osteophyte is greater than 90 mm², then the bone balance adjustment algorithm 760 may determine a combination of adjustments, such as decreasing a resection (e.g., distal femoral resection), increasing implant thickness, and/or adjusting femoral varus and/or valgus. The determinations made at steps 1512-1522 may be output on a display or other output system, for example, as a text of instructions and/or list of steps, as a chart, visually on a virtual bone model, etc. In some examples, the determinations and/or finalized procedure plan may be output as a set of programmed instructions configured for execution by a robotic platform 640 (e.g., CNC instructions) that may assist with bone cuts or other steps in a medical procedure.

The method 1500 may include a step 1524 of placing trial implants and assessing stability and range of motion. Step 1524 may be performed intraoperatively after bone cuts are made, and further adjustments may be made intraoperatively. Although the method 1500 describes a knee surgery, aspects disclosed herein may be used to make adjustments to other joints (e.g., elbows, hands, spine, ankles, feet, neck, etc.), especially other joints where osteophytes or other growths may form and/or may need to be removed (e.g., to increase mobility and/or decrease pain or impingement).

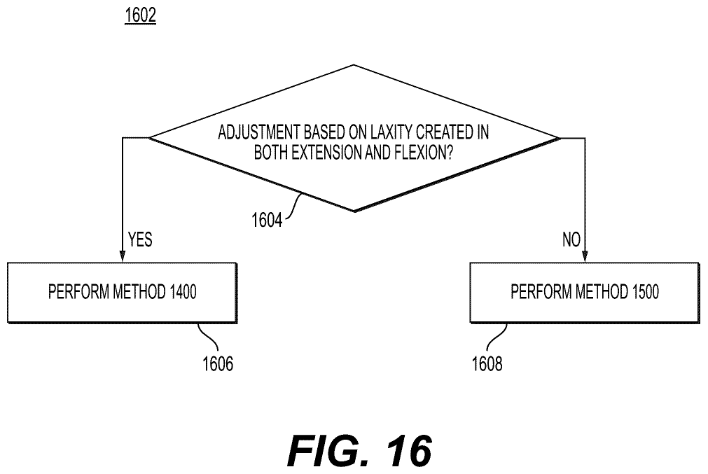

Referring to FIG. 16, the bone balance adjustment algorithm 760 may be adjusted based on whether the practitioner believes or expects osteophyte removal to affect laxity in both extension and flexion or primarily in extension. Alternatively or in addition thereto, the bone balance adjustment algorithm 760 may include a first algorithm and a second algorithm, and the bone balancing system 600 may execute the first algorithm if the practitioner believes or expects osteophyte removal to affect laxity in both extension and flexion and execute the second algorithm if the practitioner believes or expects osteophyte removal to affect laxity primarily in extension. A method 1600 may include a step 1602 of determining whether an adjustment should be made based on a prediction that osteophyte removal will create laxity in both extension and flexion. Step 1602 may include receiving input from a practitioner by, for example, providing a notification and/or graphical user interface element (e.g., pop up notification) to a practitioner for input (e.g., via display 630) and receiving the input. If, in step 1602, it is determined that adjustment should be based on laxity created in both extension and flexion (“Yes” after step 1602), the method 1400 described with FIG. 14 may be performed. If, in step 1602, it is determined that adjustment should not be based on laxity created in both extension and flexion and/or that adjustment should be based on laxity created primarily in extension (“No” after step 1602), the method 1500 described with FIG. 15 may be performed. In some examples, step 1602 may be performed after certain steps of method 1400 and/or method 1500. For example, steps 1402-1414 may be performed, and step 1602 may be performed to determine how to proceed (e.g., by continuing with steps 1416-1424 or instead performing 1516-1524). As another example, steps 1502-1516 may be performed, and step 1602 may be performed to determine how to proceed (e.g., by continuing with steps 1516-1524 or instead performing steps 1416-1424). In some examples, the bone balancing system 600 may output recommendations based on determinations made in any of methods 1300, 1400, 1500, and/or 1600. In some examples, the recommendations may simply refer to a “bone” resection, and the practitioner may decide which bone. For example in the context of a knee joint, a surgeon may decide whether to make the recommended modifications to the tibia if the surgeon believes that the soft tissue laxity resulting from posterior osteophyte removal effects flexion and extension equally, or make the recommended modifications to the distal femur if the surgeon believes that the soft tissue laxity resulting from posterior osteophyte removal effects extension only.

As previously described, the removal of medial osteophytes during a medical procedure may result in the previously stretched soft tissues becoming slack, which may not fully contract to an initial state. However, other conditions, such as a posteromedial bony flare, may also contribute to stretched soft tissue and looseness of joints. While this may be corrected through a soft tissue release, such a technique requires exceptionally high surgical skill and the procedure may often lead to overcorrection of deformities such as varus deformities. As described below, a reduction osteotomy may be performed to remove the bony protrusion, such as a posteromedial bony flare, using a tibial trial as a reference. The reduction osteotomy may be done before or after the removal of a medial osteophyte and/or additional bone cuts, or as a separate procedure.

A secondary or complementary surgical step will be outlined in the following section. The following surgical step is directed at the use of robotic assistance for planning and conducting a pre-resection medial reduction osteotomy during a medical procedure (e.g. TKA or PKA) to enhance coronal plane alignment. In at least one embodiment, the surgical method may assist in correcting varus deformities.

The method may be implemented by the bone balancing system 600 (e.g., using bone balance adjustment algorithm 760) and/or the practitioner. The method may include a step of receiving one or more images of a patient's anatomy, such as one or more CT scans, ultrasound images, or X-rays. The method may include receiving the one or more images into an electronic storage and receiving one or more procedure plans including one or more bone cuts. The procedure plan may also include an implant configured to be received in a bone at the one or more bone cuts. This method may also include determining the one or more procedure plans using, for example, the preoperative data 720 described with reference to FIG. 7.

Based on the received images, the method may include image processing techniques to identify bony protrusions, such as a posteromedial bony flare, and/or other attributes of the patient's bones. However, it would be appreciated that the identification may be done by receiving input from a practitioner (e.g. via a computer). Subsequently, the method may encompass determining various characteristics of the identified bony flare, such as surface area or shape. This is followed by establishing bone resection and implant parameters. Once these initial determinations are made, the method may generally involve executing the bone balance adjustment algorithm 760, which may involve utilizing linear relationships discussed earlier in FIG. 6. The practitioner may provide input or refine the linear relationship. Additionally, the relationship may be learned or refined through the evaluation of multiple patients.

After implementing the bone balance adjustment algorithm 760, adjustments to the bone resection and implant parameters may be necessary. For example, the bone balance adjustment algorithm 760 may recommend additional bone resection and/or downsizing of the tibial component of an implant. In some embodiments, the bone balance adjustment algorithm may be configured to determine the one or more bone resection parameters and/or one or more implant parameters for a joint based on the amount of soft tissue laxity that will be created by undergoing the medial reduction osteotomy. In other embodiments, the bone balance adjustment algorithm may be configured to determine the one or more bone resection parameters and/or one or more implant parameters for a joint based on the amount of soft tissue laxity that will be created by both removing identified osteophytes (as detected in prior methods) and subsequent medial reduction osteotomy.

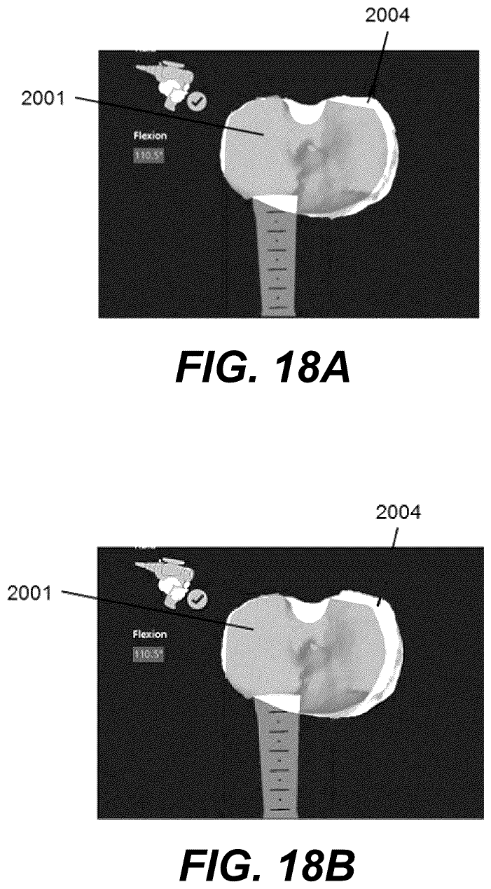

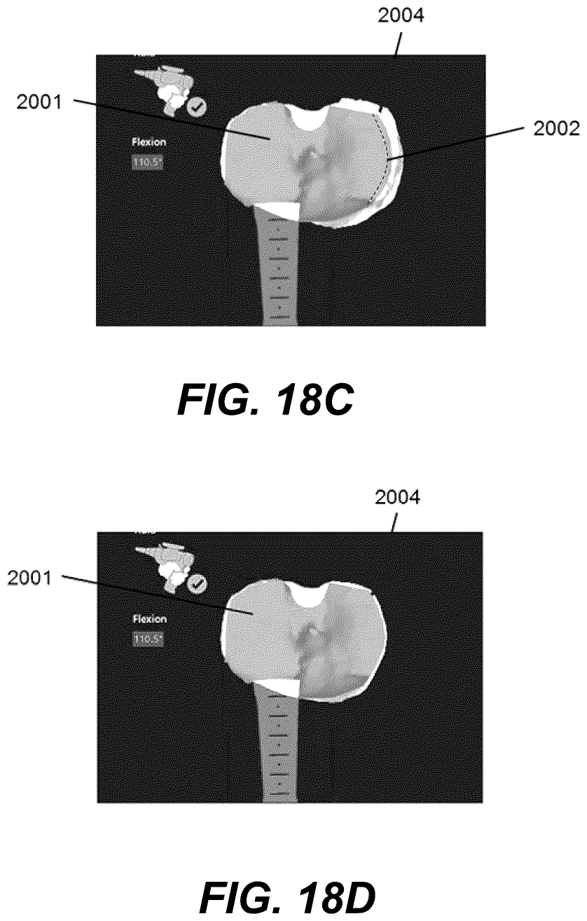

As shown in FIGS. 18A-18D, the bone balance adjustment algorithm 760 may provide visual guidance to the surgeon through a visual display. As shown in FIG. 18A, a tibial trial or component 2001 is measured against the tibial plateau 2004. If the surgeon wishes to perform a medial reduction osteotomy, then the tibial component is first lateralized, and then downsized, as shown in FIG. 18B. Then, as shown in FIG. 18C, the method may provide a guide line 2002 which indicates the necessary length and angle of a bone cut to achieve the desired shape of the bone. The guideline 2002 may be determined virtually using the parameters of a smaller sized implant projected onto the CT based image of the native pre-resected tibial plateau. The result of following the guide line 2002 is shown in FIG. 18D, which displays the post-operative bone shape. In some embodiments, the bone balance adjustment algorithm 760 may direct a robotic device to perform the bone resection on behalf of a surgeon. The decision on bone resection timing may occur intraoperatively, post-osteophyte removal, or as part of the preoperative planning process. In some embodiments, the osteotomy is conducted “pre-resection”, i.e. before any bone cuts are made.

The bone balance adjustment algorithm 760 may then determine the amount of bone needed to be resected to adjust for the laxity caused by the removal of the posteromedial bony flare (or other bone removal in the surgical plan). This step may be performed after the removal of the medial osteophyte or preoperatively. In some embodiments, the osteotomy is performed before other bony cuts, to not interfere with digital tensor function. It should be appreciated that the degree of bone resection, such as the amount of tibial bone removed during a medial reduction osteotomy, directly impacts the degree of correction of coronal plane alignment. A linear correlation exists between the extent of bone resection and degree of coronal plane correction achieved. In particular, there is about a 1° correction of the alignment of the coronal plane for about every 1 mm-2 mm of resected bone. For example, performing a medial reduction osteotomy for varus deformity may result in about a 1° correction in coronal plane alignment after the resection of 1 mm of tibial bone. In some examples, the determined coronal plane correction resulting from the planed removal of tibial bone (i.e. a medial reduction osteotomy), may be displayed pre-operatively or intra-operatively, and may be adjusted based on real-time bone resection results (e.g. detected bone resection via one or more imaging systems or practitioner input), and surgical plan adjustments may be calculated, based on the real-time bone resection results, via one or more algorithms and displayed to the practitioner.

It should be understood that steps of one or more of the various methods described herein may be combined in certain embodiments. Furthermore, in certain embodiments, fewer than all of the steps of a method described herein may be performed and/or additional steps not described herein may be performed. Moreover, the steps described herein need not necessarily be performed in the exact order presented. Additionally, although the surgical step is presented as a separate process, it should be noted that it can be seamlessly incorporated into other steps detailed in this disclosure. For instance, the adjustment of a preoperative or procedural plan to account for identified bony protrusions, like a posteromedial bony flare, may be concurrently carried out with the method 1200 for adjusting a preoperative or procedure plan based on detected osteophytes.

### Example Results

Some or all of the above described systems and methods have been implemented collecting results of osteophyte resection and ligament balancing. In the following illustrative example, 310 CAT scan based robotic Total Knee Arthroplasties (TKA) in a one year period were performed and the results collected. Pre-operative CT scans were analyzed with respect to position and dimension of inaccessible posterior osteophytes. Of the 310 patients that receives a TKA, 74 patients (24%) were noted to have posterior femoral osteophytes. Of these 74 patients, 70 knees received a knee implant without the use of cement to secure the implant (e.g., cement-less (95%)).

Of the 74 patient cases including osteophytes, 95% (e.g., 70 of 74) were associated with varus deformity. Of the 70 knees, all had medial posterior osteophytes, and 32 also had smaller lateral posterior osteophytes. Approximately 5% of the 74 patients, osteophytes were detected in valgus knees, which further included lateral posterior osteophytes, and half had smaller medial posterior osteophytes.

Size of each osteophyte was determined. To determine the size of the osteophyte, the cross sectional area of the posterior femoral osteophytes in the sagittal plane were measured with techniques consistent with the present disclosure. Medial posterior femoral osteophytes had an average size of 111 mm2, with a range of size between 32 mm2 and 321 mm2, of which 50% of the osteophytes being classified as small (under 100 mm2), 39% classified as medium (100-200 mm2), and 10% classified as jumbo (over 200 mm2). In this example data set, lateral posterior femoral osteophytes included an average size was 123 mm2, of which 46% were classified as small, 40% as medium, and 14% jumbo.

After removal of accessible osteophytes, soft tissue poses were captured, and bony balancing was performed according to the techniques described throughout the present disclosure. An assessment of sagittal alignment was then performed with further adjustment in distal femoral cut based on the presence of flexion contracture or recurvatum (+/−1 mm of distal femoral resection per 6° of deformity). A final adjustment in bony balancing is then performed based on the size and location of the posterior femoral osteophytes. In this example, laxity is adjusted based on the osteophyte correction, making changes to the tibial cut based on the size and shape of the posterior osteophytes, since the laxity created by osteophyte removal may affect both extension and flexion.

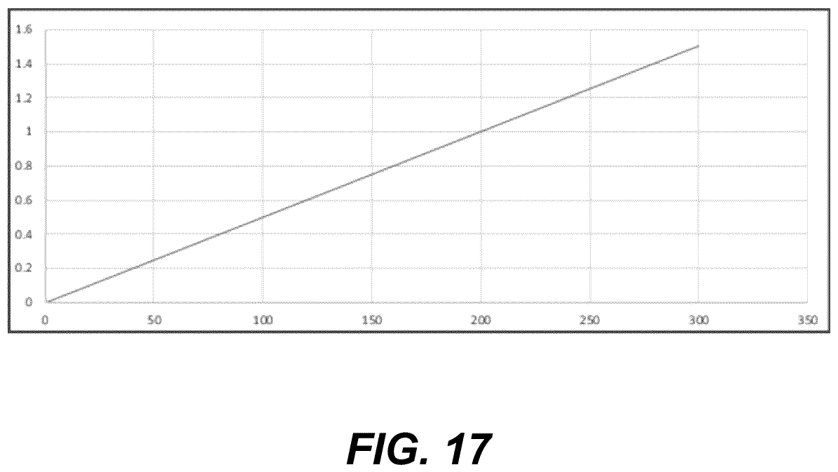

When asymmetric medial or lateral posterior osteophytes a detected, an isolated angular adjustment to the tibial resection was made in 60% ( 44/74) of knees, of which 57% ( 42/74) were varus, and 3% ( 2/74) were valgus. The average change in alignment was 0.5° (range 0.2-2.0°). When symmetric medial and lateral posterior osteophytes were detected, an isolated tibial resection level adjustment (proximalization) was made in 18% ( 13/74) of knees, with an average decrease in tibial resection of 0.85 mm (range 0.5 mm-2 mm). When asymmetric medial and lateral posterior osteophytes were detected, a combined angular and resection level correction was made to the tibia in 20% ( 15/74) of knees, of which 15% ( 11/74) were varus, and 5% ( 4/74) were valgus (see FIG. 17).

In all example cases studied where osteophytes were detected and corrected using the bony balancing algorithm (which may be any of the algorithms discussed herein), less bone was removed from the tibia 100% of the time (74/74), and an angular correction nudging alignment towards the mechanical axis was accomplished in 82% ( 61/74) knees. In some examples, a 9 or 10 mm liner was used in 99% ( 73/74) of knees (66 patients received 9 mm, 7 patients received 10 mm), and an 11 mm liner was used in one patient.

Aspects disclosed herein may provide one or more algorithms to determine one or more bone resection parameters and/or implant parameters and predict soft tissue effects (and/or gap or balance effects) after removal of one or more osteophytes and/or posterior ligament release, allowing a surgeon to automate preoperative and/or intraoperative decision making. The algorithm may help streamline a workflow for a procedure to anticipate the effect of removal of one or more osteophytes (e.g., posterior osteophyte) on soft tissue balancing, and to proactively adjust bone cuts accordingly.

Aspects disclosed herein may provide an algorithm that calculates one or more bone resection parameters and/or one or more implant parameters based on a cross-sectional or surface area of an osteophyte. The relationship may include adjusting parameters by a predetermined amount per a predetermined amount of cross-sectional area. For example, a linear relationship may include adjusting a bone resection parameter and/or implant parameter by 0.5° and/or 0.5 mm per every 90 mm² of cross-sectional area of the osteophyte, or 0.1° and/or 0.1 mm per every 20 mm² of cross-sectional area of the osteophyte, but aspects disclosed herein are not limited. For example, the predetermined amount in parameters may be in a range of 0.4°-0.6° and/or 0.4 mm-0.6 mm, and the predetermined amount of cross-sectional area may be in a range of 80 mm² or 85 mm² to 100 mm², etc. As another example, the predetermined amount in parameters may be in a range of 0.075°-0.125° and/or 0.075 mm-0.125 mm, and the predetermined amount of cross-sectional area may be in a range of 15 mm² to 25 mm², etc. The linear relationship may correspond to y=mx+b or y=mx, where “m” may be 0.0055 or 0.0056, or in a range of 0.005-0.006 or 0.004-0.006 (for example, in a context of a knee joint). For other joints, “m” and/or the linear relationship may differ based on a learned relationship.

Aspects disclosed herein may be adjusted to a practitioner's preferences and/or methods. For example, some practitioners prefer to create rectangular gaps and/or bone resections, while other practitioners prefer to create trapezoidal gaps and/or bone resection by, for example, making a lateral side looser and/or using a greater bone slope at the lateral side. Aspects disclosed herein may be used to adjust an initial procedure plan that was generated according to the practitioner's preferences, and adjustments themselves may be tweaked based on practitioner preference.

Aspects disclosed herein may assist practitioners in planning bone cuts for patients undergoing a joint replacement surgery to correct a varus or valgus deformity, where removal of posterior osteophytes may significantly affect laxity and/or a surgery result. Aspects disclosed herein may consider an extent of deformity in determining modifications to bone resection parameters and/or implant thickness to account for osteophyte removal.

Aspects disclosed herein may provide an algorithm that predicts increases in an extension gap and flexion gap (or, alternatively, an extension gap) after removal of one or more posterior osteophytes based on a cross-sectional area of the one or more posterior osteophytes. Aspects disclosed herein may provide recommendations based on a correlation between osteophyte size and/or location and soft tissue affect.

Aspects disclosed herein may simplify a prediction of a soft tissue effect caused by osteophyte removal by considering a cross-sectional area at a largest part of the osteophyte (e.g., using one slice of the CT scan) and by not requiring more dimensions and/or calculations (e.g., an osteophyte volume). Aspects disclosed herein may not require consideration of a width of the osteophyte and/or an entire volume of the osteophyte.

Aspects disclosed herein may provide recommendations regardless of whether a deformity is fixed and/or flexible, and may not require input of whether a deformity is fixed or flexible. Aspects disclosed herein make predictions on an effect on soft tissue laxity that is independent of a rigidity of the deformity. Aspects disclosed herein may provide a linear correlation between a size (e.g., surface area and/or cross-sectional area) of an osteophyte removed and a resultant soft tissue laxity. Additionally, and/or alternatively, aspects disclosed herein may provide a non-linear correlation between size of an osteophyte removed and a resultant soft tissue laxity.

Aspects disclosed herein may provide recommendations regardless of patient reported outcomes, and may not require input of patient reported outcomes. Aspects disclosed herein may correlate outcome with osteophyte size based on a correlation between osteophyte volume and disease severity or progression. Aspects disclosed herein may predict a greater outcome based on removal of a larger osteophyte.

Aspects disclosed herein may identify a direction and a magnitude of a deformity and an anticipated result of osteophyte removal. Aspects disclosed herein may provide a predictive algorithm and/or provide granular detail to identify a direction and a magnitude of a deformity to account for posterior osteophyte removal and subsequent increase in an extension and/or flexion gap. Aspects disclosed herein may provide an algorithm configured to characterize ac scope of deformity both in direction and magnitude.

Aspects disclosed herein may be used to sense or collect preoperative, intraoperative, and/or postoperative information about a patient and/or a procedure.

Aspects disclosed herein contemplate implants or prosthetics, and are not limited to the contexts described. For example, implants disclosed herein may be implemented as another implant system for another joint or other part of a musculoskeletal system (e.g., hip, knee, spine, bone, ankle, wrist, fingers, hand, toes, or elbow) and/or as sensors configured to be implanted directly into a patient's tissue, bone, muscle, ligaments, etc. Each of the implants or implant systems may include sensors such as inertial measurement units, strain gauges, accelerometers, ultrasonic or acoustic sensors, etc. configured to measure position, speed, acceleration, orientation, range of motion, and/or sensors configured to measure biometric changes (e.g., color change, pH change, etc.) in synovial fluid, blood glucose, temperature, or other biometrics and/or may include electrodes that detect current information, ultrasonic or infrared sensors that detect other nearby structures, etc. to detect an infection, invasion, nearby tumor, etc. The implants may, for example, be a sensor or other measurement device configured to be drilled into a bone, another implant, or otherwise implanted in the patient's body.

Aspects and systems disclosed herein may make determinations based on images or imaging data (e.g., from CT scans, ultrasounds). Aspects disclosed herein may predict soft tissue laxity, a resulting gap, bone resection and/or implant parameters, etc. based on one slice or view of a CT scan and may not require calculating an osteophyte volume, etc. Images disclosed herein may display or represent bones, tissues, or other anatomy, and systems and aspects disclosed herein may recognize, identify, classify, and/or determine portions of anatomy such as bones, cartilage, tissue, and bone landmarks, such as each specific vertebra in a spine. Aspects and systems disclosed herein may determine relative positions, orientations, and/or angles between recognize bones, such as a Cobb angle, an angle between a tibia and a femur, and/or other alignment data.

Aspects and systems disclosed herein provide displays having graphical user interfaces configured to graphically display data, determinations, and/or steps, targets, instructions, or other parameters of a procedure, including preoperatively, intraoperatively, and/or postoperatively. Figures, illustrations, animations, and/or videos displayed via user interfaces may be recorded and stored on the memory system.

Aspects and systems disclosed herein may be implemented using machine learning technology. One or more algorithms may be configured to learn or be trained on patterns and/or other relationships across a plurality of patients in combination with preoperative information and outputs, intraoperative information and outputs, and postoperative information and outputs. The learned patterns and/or relationships may refine determinations made by one or more algorithms and/or also refine how the one or more algorithms are executed, configured, designed, or compiled. The refinement and/or updating of the one or more algorithms may further refine displays and/or graphical user interfaces (e.g., bone recognition and/or determinations, targets, recognition and/or display of other conditions and/or bone offsets, etc.).

Aspects disclosed herein may be configured to optimize a “fit” or “tightness” of an implant provided to a patient during a medical procedure based on detections by the one or more algorithms. A fit of the implant may be made tighter by aligning the implant with a shallower bone slope and/or determining a shallower resulting or desired bone slope, by increasing a thickness or other dimensions of the implant, by determining certain types of materials or a type of implants or prosthesis (e.g., a stabilizing implant, a VVC implant, an ADM implant, or an MDM implant). A thickness of the implant may be achieved by increasing (or decrease) a size or shape of the implant. Tightness may be impacted by gaps and/or joint space width, which may be regulated by an insert which may vary depending on a type of implant or due to a motion. Gaps may be impacted by femoral and tibial cuts. Tightness may further be impacted by slope. A range of slope may be based on implant choice as well as surgical approach and patient anatomy. A thickness of the implant may also be achieved by adding or removing an augment or shim. For example, augments or shims may be stackable and removable, and a thickness may be increased by adding one or more augments or shims or adding an augment or shim having a predetermined (e.g., above a certain threshold) thickness. Fit or tightness may also be achieved with certain types of bone cuts, bone preparations, or tissue cuts that reduce a number of cuts made and/or an invasiveness during surgery.

Aspects disclosed herein may be implemented during a robotic medical procedure using a robotic device. Aspects disclosed herein are not limited to specific scores, thresholds, etc. that are described. For example, outputs and/or scores disclosed herein may include other types of scores such as the hip disability and osteoarthritis score or HOOS, KOOS, SF-12, SF-36, Harris Hip Score, etc.

Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgery outcomes, implant designs, and/or preoperative analyses, predictions, or workflows. Aspects disclosed herein may augment the continuum of care to optimize post-operative outcomes for a patient. Aspects disclosed herein may recognize or determine previously unknown relationships, to help optimize care, predict soft tissue laxity, and/or to optimize design of a prosthetic or implant.

What is claimed is:

1. A method of assessing a joint, comprising:
identifying a first osteophyte in an image of the joint, wherein the first osteophyte is positioned under a soft tissue;
identifying a cross-sectional area of the first osteophyte;
executing an algorithm to determine one or more adjustment parameters based on the identified cross-sectional area of the first osteophyte, wherein the algorithm applies an equation that receives, as input, the identified cross-sectional area, and outputs the one or more adjustment parameters, wherein the one or more adjustment parameters include:
a predicted change in soft tissue laxity after the identified first osteophyte is removed;
an adjustment to a planned bone resection depth of the one or more bone cuts;
an adjustment to a planned bone resection angle of the one or more bone cuts; and/or
an adjustment to a planned thickness of the implant; and
outputting the one or more determined adjustment parameters to a display.

2. The method of claim 1, wherein identifying the first osteophyte and/or identifying the cross-sectional area of the first osteophyte includes analyzing the image using one or more image processing techniques.

3. The method of claim 1, wherein identifying the cross-sectional area of the first osteophyte includes analyzing a first dimension of the first osteophyte and a second dimension of the first osteophyte, and disregarding a third dimension of the first osteophyte.

4. The method of claim 1, wherein the one or more adjustment parameters include an adjustment to a planned bone resection depth of the one or more bone cuts and an adjustment to a planned bone resection angle of the one or more bone cuts.

5. The method of claim 1, wherein identifying the cross-sectional area of the first osteophyte includes determining that the image of the joint is an image of a set of images showing a greatest extent of the first osteophyte in a first dimension.

6. The method of claim 1, wherein the equation is a linear equation, the linear equation includes:
a linear relationship between the identified cross-sectional area and the adjustment to the planned bone resection depth such that, the greater the identified cross-sectional area, the greater the decrease in planned bone resection depth; and/or
a linear relationship between the identified cross-sectional area and the planned thickness of the implant such that, the greater the identified cross-sectional area, the greater the increase to the planned thickness of the implant.

7. The method of claim 1, further comprising:
identifying a second osteophyte in the image of the joint that is positioned under the soft tissue;
identifying a cross-sectional area of the second osteophyte;
identifying a position of the first osteophyte; and
identifying a position of the second osteophyte;
wherein executing the algorithm to determine the one or more adjustment parameters is further based on the identified cross-sectional area of the second osteophyte, the identified position of the first osteophyte, and the identified position of the second osteophyte.

8. The method of claim 7, further comprising:
determining, based on the identified position of the first osteophyte and the identified position of the second osteophyte, the first osteophyte is provided at a first side of the joint and the second osteophyte is provided at a second side of the joint opposite the first side; and
determining a difference in the identified cross-sectional area of the first osteophyte and the identified cross-sectional area of the second osteophyte, wherein the equation includes a linear relationship between the determined difference in the identified cross-sectional areas and the adjustment to the planned bone resection angle such that, the greater the determined difference in the identified cross-sectional areas, the greater the adjustment to the planned bone resection angle.

9. The method of claim 8, further comprising determining whether a difference in the identified cross-sectional areas is greater than or equal to a predetermined difference threshold, wherein:
if the determined difference in the identified cross-sectional area is not greater than or equal to the predetermined difference threshold, determining that the first osteophyte and the second osteophyte are symmetric; and
if the determined difference in the identified cross-sectional area is greater than the predetermined difference threshold, determining that the first osteophyte and the second osteophyte are asymmetric.

10. The method of claim 9, wherein:
if the first osteophyte and the second osteophyte are determined to be symmetric, then executing the algorithm includes:
determining that the planned bone resection depth should be decreased by a predetermined amount of depth per a predetermined amount of the identified cross-sectional area of the first osteophyte and/or the second osteophyte, or
determining that the planned thickness of the implant should be increased by the predetermined amount of depth per the predetermined amount of the identified cross-sectional area of the first osteophyte and/or the second osteophyte; and
if the first osteophyte and the second osteophyte are determined to be asymmetric, then executing the algorithm includes:
determining, based on the identified cross-sectional area of the first osteophyte and the identified cross-sectional area of the second osteophyte, whether the first osteophyte is larger than the second osteophyte;
if the first osteophyte is determined to be larger than the second osteophyte, determining that the planned bone resection angle should be adjusted in a first direction or orientation by a predetermined amount of bone resection angle per a predetermined amount of difference between the identified cross-sectional area of the first osteophyte and the identified cross-sectional area of the second osteophyte;
if the first osteophyte is determined not to be larger than the second osteophyte, determining that the second osteophyte is larger than the first osteophyte, and determining that the planned bone resection angle should be adjusted in a second direction or orientation opposite the first direction or orientation by the predetermined amount of bone resection angle per the predetermined amount of difference.

11. The method of claim 10, further comprising:
determining that the first osteophyte and the second osteophyte are asymmetric;
determining that both the identified cross-sectional area of the first osteophyte and the identified cross-sectional area of the second osteophyte are greater than a predetermined cross-sectional area,
determining that the difference in the identified cross-sectional areas is greater than a predetermined difference,
determining that the planned bone resection depth should be decreased by the predetermined amount of depth per the predetermined amount of the identified cross-sectional area of the first osteophyte and/or the second osteophyte, or
determining that the planned thickness of the implant should be increased by the predetermined amount of depth per the predetermined amount of the identified cross-sectional area of the first osteophyte and/or the second osteophyte.

12. The method of claim 10, wherein the joint is a knee joint, and:
the first osteophyte is a lateral osteophyte and the second osteophyte is a medial osteophyte;
the one or more bone cuts includes a tibial bone cut or a femoral bone cut;
the first direction is a tibial varus or a femoral varus; and
the second direction is a tibial valgus or a femoral valgus.

13. The method of claim 12, wherein:
the predetermined amount of depth is in a range of 0.4 millimeters (mm) to 0.6 mm;
the predetermined amount of the identified cross-sectional area of the first osteophyte and/or the second osteophyte is in a range of 85 mm² to 100 mm²;
the predetermined amount of bone resection angle is in a range of 0.4° to 0.6°;
the predetermined amount of difference between the identified cross-sectional area of the first osteophyte and the identified cross-sectional area of the second osteophyte is in a range of 80 mm² to 100 mm².

14. The method of claim 12, wherein:
the predetermined amount of depth is in a range of 0.05 mm-1.5 mm;
the predetermined amount of the identified cross-sectional area of the first osteophyte and/or the second osteophyte is in a range of 15 mm² to 25 mm²;
the predetermined amount of bone resection angle is in a range of 0.05°-1.5°,
the predetermined amount of difference between the identified cross-sectional area of the first osteophyte and the identified cross-sectional area of the second osteophyte is in a range of 15 mm² to 25 mm².

15. The method of claim 1, wherein the equation includes a non-linear relationship.

16. A method of assessing a joint, comprising:
receiving an image of a joint including an osteophyte, wherein the image shows a view of the joint in a first dimension and a second dimension over which soft tissue extends over the osteophyte;
receiving a procedure plan including a plan to remove the osteophyte after one or more bone cuts are made, wherein the one or more bone cuts are configured for installation of an implant during the procedure;
identifying a cross-sectional area of the osteophyte in the first dimension and the second dimension;
executing an algorithm to determine one or more adjustment parameters, wherein the algorithm applies an equation that receives, as input, the identified cross-sectional area of the at least one osteophyte, and outputs the one or more adjustment parameters, wherein the one or more adjustment parameters include an adjustment to one or more bone resection parameters and/or an adjustment to an implant parameter of the received procedure plan; and
outputting the one or more determined adjustment parameters to a display.

17. The method of claim 16, wherein the one or more bone resection parameters include a planned bone resection depth of the one or more bone cuts and/or a planned bone resection angle of the one or more bone cuts.

18. A system configured to assess a joint, comprising:
an image acquisition device configured to acquire at least one image of the joint;
a memory configured to store information, the information including imaging data related to the at least on acquired image, wherein the imaging data includes a cross-sectional area and position of at least one identified portion of the bone of the joint;
a controller configured to:
execute an algorithm to determine, based on the at least one acquired image and/or the stored imaging data, one or more adjustment parameters, wherein the algorithm applies an equation that receives, as input, the cross-sectional area of the at least one identified portion of the bone, and outputs the one or more adjustment parameters, wherein the one or more adjustment parameters include an adjustment to a bone resection parameter and/or an adjustment to an implant parameter; and
a display configured to display the determined one or more adjustment parameters.

19. The system of claim 18, wherein the image acquisition device is a computed tomography (CT) acquisition device, and the acquired at least one image is a CT scan; and
wherein the CT scan shows a view of the joint in a first dimension and a second dimension over which the soft tissue extends, and the cross-sectional area is determined using the dimension of the identified portion of the bone in the first dimension and the second dimension.

20. The system of claim 18, wherein the one or more adjustment parameters includes a coronal plane alignment, and wherein the bone resection parameter is used to determine the coronal plane alignment.
